(19) United States
(12) Patent Application Publication
(10) Pub. No.: US 2022/0273442 A1
(43) Pub. Date: Sep. 1, 2022

(54) BONE TIE METHODS

(71) Applicant: Spinal Elements, Inc., Carlsbad, CA (US)

(72) Inventors: Jason Blain, Encinitas, CA (US); Taylor Semingson, San Diego, CA (US); Megan Monier, Vista, CA (US)

(21) Appl. No.: 17/681,575

(22) Filed: Feb. 25, 2022

Related U.S. Application Data
(60) Provisional application No. 63/154,959, filed on Mar. 1, 2021.

(51) Int. Cl. A61F 2/30; A61F 2/44; A61B 17/84

(52) U.S. Cl. CPC A61F 2/30749; A61F 2/4455; A61B 17/842; A61F 2002/30772; A61F 2002/2835

(57) ABSTRACT

Various embodiments of bone ties, interbody devices, and methods for treating the spine are provided. The method can include positioning an interbody device in an intervertebral space. The method can include positioning the bone tie through the interbody device. The method can include tightening the bone tie, wherein the bone tie is configured to promote spinal fusion.

### CROSS-REFERENCE TO RELATED APPLICATIONS

This application claims priority benefit to U.S. Provisional Patent Application No. 63/154,959, filed Mar. 1, 2021, the entirety of which is hereby incorporated by reference herein.

### FIELD

Some embodiments described herein relate generally to systems and methods for performing spinal fusion and, in particular, to bone ties and interbody devices.

### DESCRIPTION OF THE RELATED ART

Advancing age, as well as injury, can lead to degenerative changes in the bones, discs, joints and ligaments of the spine, producing pain and instability. Under certain circumstances, alleviation of the problems can be provided by performing spinal fusion. Spinal fusion is a surgical technique where two or more vertebrae of the spinal column are fused together to eliminate the motion between the fused vertebrae. Spinal fusion is used to treat conditions where the spine exhibits instability. Spine instability may result from causes such as fracture, scoliosis and spondylolisthesis, where one or more vertebrae move in a forward direction relative to the other vertebrae. Spinal fusion with discectomy is also performed for herniations of the discs. This surgery involves removal of the affected disc and fusion of the adjacent vertebrae. Traditionally, bone grafts have been used to fuse the vertebrae, but various types of vertebral implants have also been used.

The use of bone plate and bone screw fixation systems for treating injuries to bones is well established. In most instances, a bone plate is positioned over and surrounding the bone injury area and secured to the bone. The bone plate is secured to the bone using bone screws or other similar fasteners inserted through holes in the bone plate and into the bone itself. The screws are tightened so that the bone plate holds the bone to be treated in place in order to insure proper healing. Early fixation devices tended to be applicable only to long bone injuries with only limited uses for lower lumbar spinal injuries and disorders. The use of plate/screw fixation systems later expanded, however, to include more uses for spinal injuries, including fusion of vertebrae including fixation devices for treating vertebrae injuries. Notwithstanding the foregoing, there remains a need for improved methods and devices for treating spinal instability.

### SUMMARY

Devices and methods are disclosed for treating the vertebral column. In some embodiments, a bone tie for securing or fusing vertebrae is provided. The bone ties and the interbody devices can be used to stabilize and/or fixate a first vertebra to a second vertebra. The bone ties and the interbody devices can be used to reduce the pain. The bone ties and the interbody devices can be used to reduce further degradation of a spine. The bone ties and the interbody devices can be used to stabilize or fixate a specific vertebra of a spine. The bone ties and the interbody devices can be used to fuse the first vertebra and the second vertebra.

In some embodiments, a method of treating a patient is provided. The method can include positioning an interbody device in an intervertebral space. The method can include positioning a bone tie through the interbody device, the bone tie comprising a distal end and a fastener section. The method can include tightening the bone tie by passing the distal end of the bone tie through the fastener section of the bone tie.

In some embodiments, the method can include forming a lumen in a vertebra. In some embodiments, the method can include positioning the bone tie through the lumen. In some embodiments, the method can include forming a lumen in a superior vertebra and an inferior vertebra. In some embodiments, the method can include positioning the bone tie through the lumen in the superior vertebra. In some embodiments, the method can include positioning a second bone tie through the interbody device and through the lumen in the inferior vertebra. In some embodiments, positioning the bone tie through the interbody device further comprises passing the bone tie through a graft chamber. In some embodiments, positioning the bone tie through the interbody device comprises passing the bone tie through a window. In some embodiments, the method can include packing a window of the interbody device with graft material. In some embodiments, the bone tie is in communication with graft material. In some embodiments, the bone tie is not in communication with graft material. In some embodiments, positioning the bone tie through the interbody device comprises passing the bone tie through a notch. In some embodiments, positioning the bone tie through the interbody device comprises passing the bone tie toward a lumen in a vertebra. In some embodiments, tightening the bone tie comprises engaging gears of a bone tie with a ratchet. In some embodiments, the interbody device is retained within a loop of the bone tie. In some embodiments, tightening the bone tie comprises pulling the distal end through the fastener section to make a loop consecutively smaller. In some embodiments, the method can include packing the interbody device with graft material. In some embodiments, the method can include packing the interbody device after the bone tie is positioned through the interbody device. In some embodiments, the method can include packing the interbody device before the bone tie is positioned through the interbody device.

In some embodiments, a kit is provided. The kit can include a bone tie comprising a distal end and a fastener section. In some embodiments, the bone tie is configured to form a loop by passing the distal end through the fastener section. The kit can include an interbody device comprising a notch or a window configured to receive the bone tie. In some embodiments, the interbody device is configured to be retained within the loop of the bone tie.

In some embodiments, the bone tie is monolithically formed. In some embodiments, the interbody device comprises a notch extending from a proximal end to a superior surface. In some embodiments, the interbody device comprises a notch extending from a superior surface to an inferior surface. In some embodiments, the interbody device comprises a notch in communication with a window. In some embodiments, the interbody device comprises a notch closed toward a window. In some embodiments, the interbody device comprises a first notch extending from a proximal end to a superior surface and a second notch extending from the proximal end to an inferior surface. In some embodiments, the interbody device comprises a window configured to be packed with graft material.

In some embodiments, an apparatus is provided. The apparatus can include an interbody device comprising at least one lumen sized for passage of a bone tie. In some embodiments, the at least one lumen is located near an edge of the interbody device. In some embodiments, the at least one lumen is accessible after implantation and configured to secure the interbody device to bone.

In some embodiments, the at least one lumen comprises a notch extending from a superior surface to an inferior surface. In some embodiments, the at least one lumen is in communication with a graft window. In some embodiments, the at least one lumen is not in communication with a graft window. In some embodiments, the at least one lumen comprises a notch extending from a proximal end to a superior surface. In some embodiments, the at least one lumen comprises a first notch extending from a proximal end to a superior surface and a second notch extending from the proximal end to an inferior surface. In some embodiments, the at least one lumen comprises a notch extending from a side surface. In some embodiments, the at least one lumen comprises a notch extending from an end surface. In some embodiments, the at least one lumen comprises a linear lumen. In some embodiments, the at least one lumen comprises a non-linear lumen. In some embodiments, the at least one lumen comprises a lumen angled toward a vertebral body after implantation. In some embodiments, the at least one lumen comprises a window configured to be packed with graft material. In some embodiments, the at least one lumen comprises a notch comprising a rectangular cross-sectional dimension.

### BRIEF DESCRIPTION OF THE DRAWINGS

The structure and method of use will be better understood with the following detailed description of embodiments, along with the accompanying illustrations, in which:

FIG. 1 is a lateral view of a portion of the vertebral column.

FIG. 2 is a perspective front view of an embodiment of a bone tie.

FIG. 3 is a perspective back view of the bone tie of FIG. 2.

FIG. 4 is a perspective view of a proximal portion of the bone tie of FIG. 2.

FIG. 5 is a perspective view of a distal portion of the bone tie of FIG. 2.

FIG. 6 is an enlarged perspective view of a distal portion of the bone tie of FIG. 2.

FIG. 7 is a perspective view of an embodiment of a bone tie.

FIG. 8 is a perspective view of the bone tie of FIG. 7.

FIG. 9 is a perspective view of an embodiment of a bone tie.

FIG. 10 is a side cross-sectional view of the bone tie of FIG. 9.

FIG. 11 is a top view of the bone tie of FIG. 9.

FIG. 12 is a view of an embodiment of an interbody device.

FIG. 13 is a cross-sectional view of the interbody device of FIG. 12.

FIG. 14 is a view of an embodiment of an interbody device.

FIG. 15 is a cross-sectional view of the interbody device of FIG. 14.

FIG. 16 is a view of an embodiment of an interbody device.

FIG. 17 is a cross-sectional view of the interbody device of FIG. 16.

FIG. 18 is a side view of an embodiment of a bone tie and an interbody device.

FIG. 19 is a view of an embodiment of an interbody device.

FIG. 20 is a view of an embodiment of an interbody device.

FIG. 21 is a perspective view of an embodiment of an interbody device.

FIG. 22 is a view of an interbody guide post.

FIG. 23 is a front view of a drill.

FIG. 24 is a cross-sectional view of the drill of FIG. 23.

FIG. 25 is a view of internal components of the drill of FIG. 23.

FIG. 26 is a proximal view of the drill of FIG. 23.

FIG. 27 is a distal view of the drill of FIG. 23.

FIG. 28 is a perspective view of the drill of FIG. 23.

FIG. 29 is a front view of a drill bit.

FIG. 30 is a distal view of the drill bit of FIG. 29.

FIG. 31 is a perspective view of the drill of FIG. 23 and the drill bit of FIG. 29.

FIG. 32 is a distal view of the drill of FIG. 23 and the drill bit of FIG. 29.

FIGS. 33A-33D are views of methods.

FIG. 34 is a view of a method.

### DETAILED DESCRIPTION

Although certain preferred embodiments and examples are disclosed below, it will be understood by those in the art that the disclosure extends beyond the specifically disclosed embodiments and/or uses of the invention and obvious modifications and equivalents thereof. Thus, it is intended that the scope should not be limited by the particular disclosed embodiments described below.

The systems and methods described herein relate to embodiments of bone ties, embodiments of interbody devices, and methods of use. The methods can include passing the bone tie through an interbody device. The methods can include passing the bone tie through a lumen in a portion of a vertebra. The methods can include wrapping the bone tie around a portion of a vertebra. The methods can include fusing two or more vertebrae.

1. Anatomy of the Spine

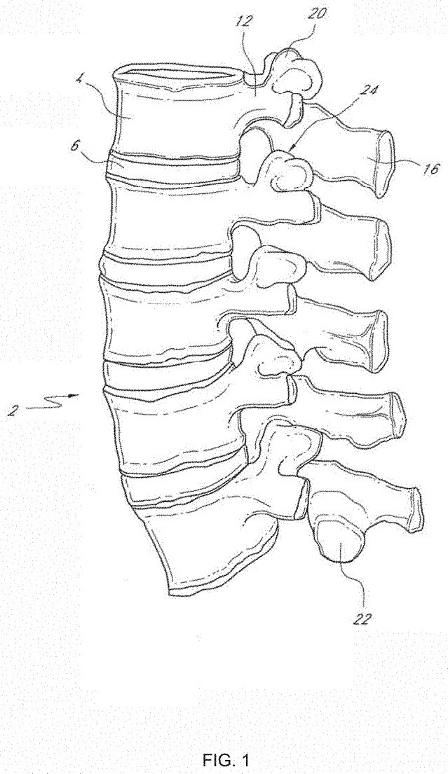

As shown in FIG. 1, the vertebral column 2 comprises a series of alternating vertebrae 4 and fibrous discs 6 that provide axial support and movement to the upper portions of the body. The vertebral column 2 typically comprises thirty-three vertebrae 4, with seven cervical (C1-C7), twelve thoracic (T1-T12), five lumbar (L1-15), five fused sacral (S1-S5) and four fused coccygeal vertebrae. Each thoracic vertebra includes an anterior body with a posterior arch. The posterior arch comprises two pedicles 12 and two laminae that join posteriorly to form a spinous process 16. Projecting from each side of the posterior arch is a transverse, superior 20 and inferior articular process 22. The facets 24 of the superior 20 and inferior articular processes 22 form facet joints with the articular processes of the adjacent vertebrae. The facet joints are true synovial joints with cartilaginous surfaces and a joint capsule.

The typical cervical vertebrae differ from the other vertebrae with relatively larger spinal canal, oval shaped vertebral bodies, bifid spinous processes and foramina in their transverse processes. These foramina transversaria contain the vertebral artery and vein. The first and second cervical vertebrae are also further differentiated from the other vertebrae. The first cervical vertebra lacks a vertebral body and instead contains an anterior tubercle. Its superior articular facets articulate with the occipital condyles of the skull and are oriented in a roughly parasagittal plane. The cranium is able to slide forward and backwards on this vertebra. The second cervical vertebra contains an odontoid process, or dens, which projects superiorly from its body. It articulates with the anterior tubercle of the atlas, forming a pivot joint. Side to side movements of the head occur at this joint. The seventh cervical vertebra is sometimes considered atypical since it lacks a bifid spinous process.

2. Bone Tie

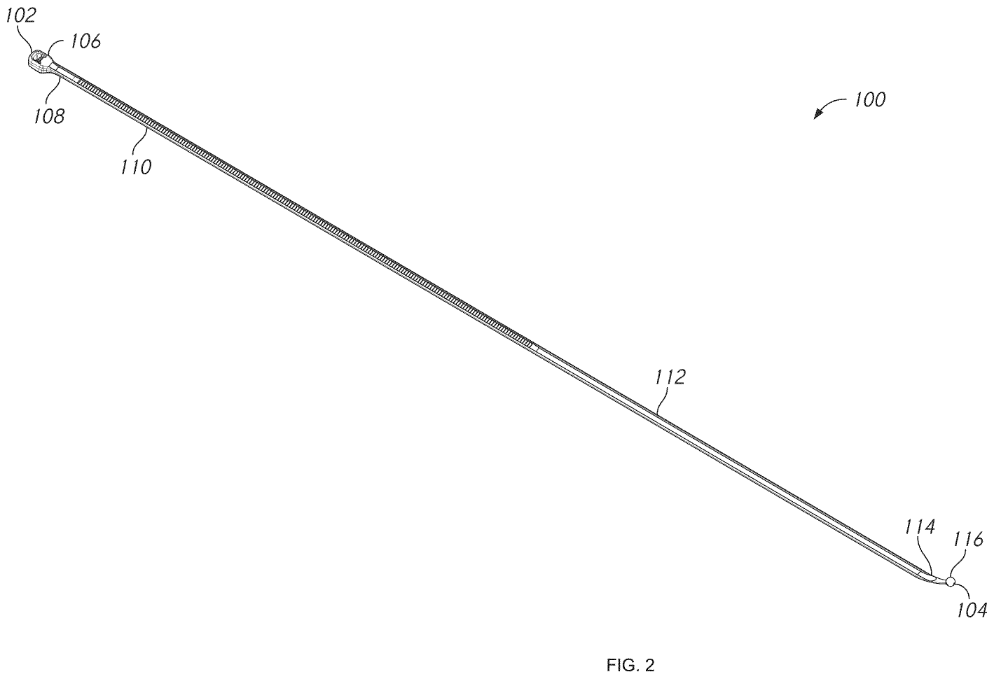

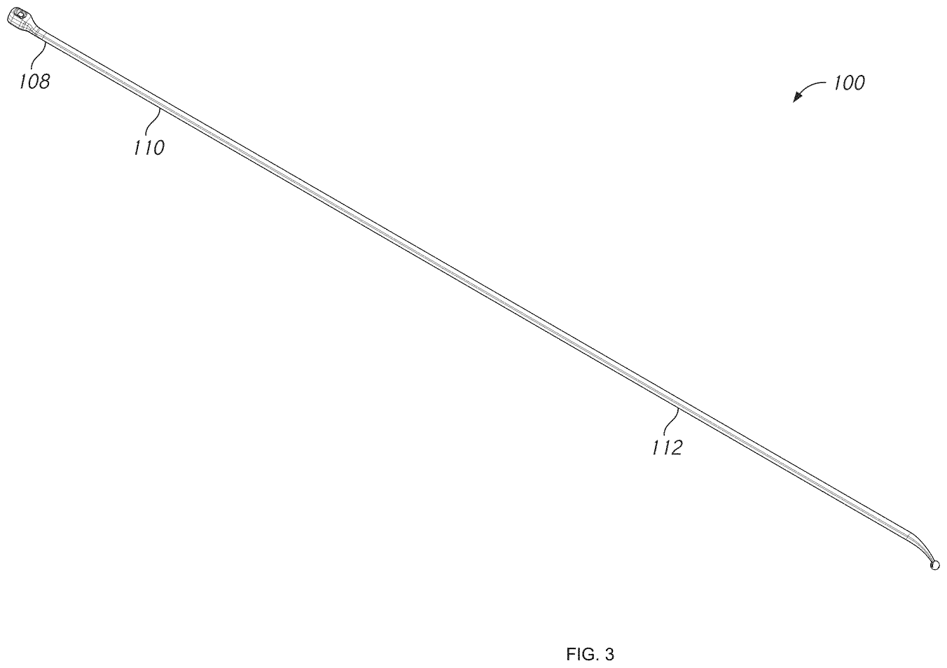

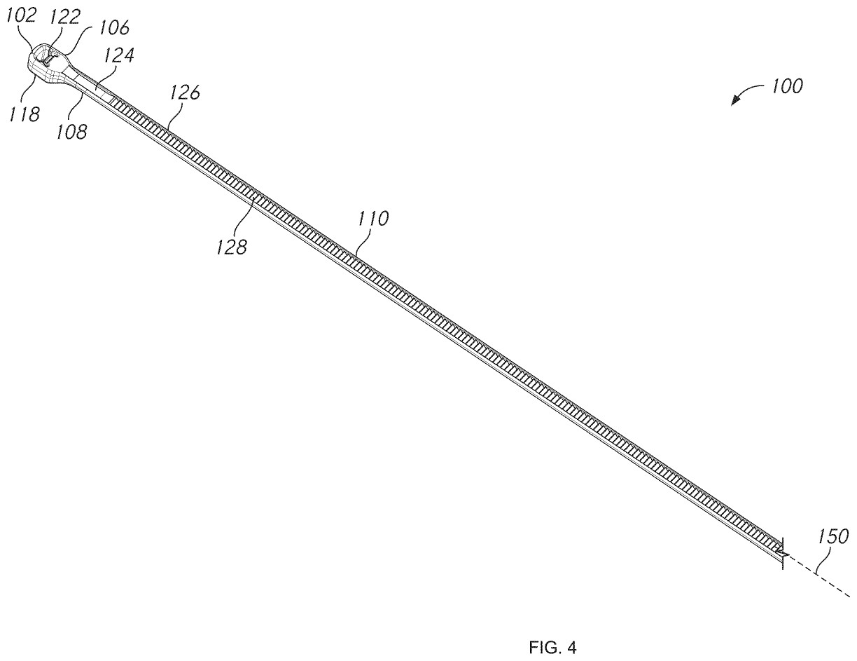

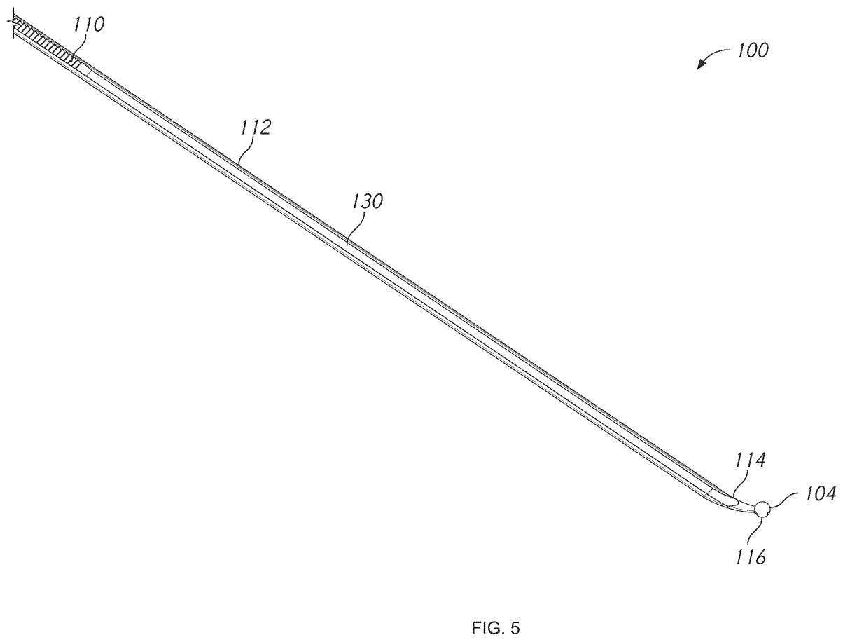

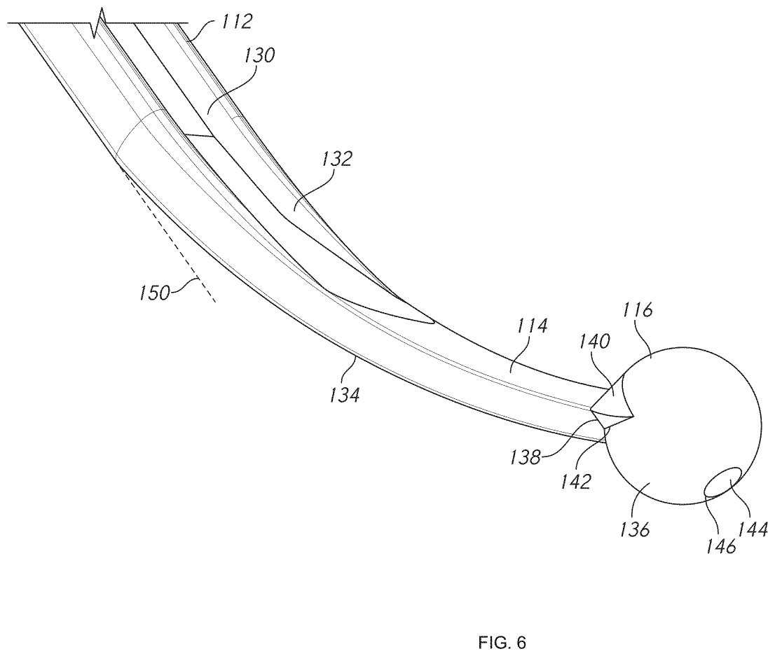

FIGS. 2-11 depict views of embodiments of bone ties. FIG. 2 illustrates a perspective front view of a bone tie 100. FIG. 3 illustrates a perspective back view of the bone tie 100. FIG. 4 illustrates a perspective view of a proximal portion of the bone tie 100. FIG. 5 illustrates a perspective view of a distal portion of the bone tie 100. FIG. 6 illustrates an enlarged perspective view of a distal portion of the bone tie 100.

FIG. 2 is a perspective front view of the bone tie 100. The bone tie 100 can be a generally elongate member. The bone tie 100 can comprise a proximal end 102 and a distal end 104. The bone tie 100 can include a length between the proximal end 102 and the distal end 104. The proximal end 102 can be configured to be near the hands of the user when the user is manipulating a bone tie inserter. The distal end 104 can be configured to be inserted into a bone lumen as described herein. The distal end 104 can be configured to be the first portion of the bone tie 100 that is inserted in the lumen. The distal end 104 can be the leading end of the bone tie 100. In some methods of use, the proximal end 102 extends away from the vertebrae during insertion of the bone tie 100. In some methods of use, the proximal end 102 is held by the user. In some methods of use, the proximal end 102 is unconstrained during insertion of the bone tie 100. In some methods of use, bone tie 100 can be grasped and manipulated by a user.

The bone tie 100 can include one or more sections along the length of the bone tie 100. The sections can have a different shape, configuration, or function than an adjacent section of the bone tie 100. In some embodiments, one or more non-adjacent sections can have the same shape, configuration, or function as another section of the bone tie 100. In some embodiments, one or more additional sections are provided. In some embodiments, one or more of the sections provided herein are omitted.

The bone tie 100 can include a fastener section 106. The fastener section 106 can be located at or near the proximal end 102. The fastener section 106 can include any mechanism configured to secure the fastener section 106 to another section of the bone tie 100. The fastener section 106 can include a mechanism that allows the bone tie 100 to be secured in a single direction of travel such as a ratchet. The fastener section 106 can include a mechanism that allows the bone tie 100 to be secured in two directions of travel such as a pair of gears.

The bone tie 100 can include a first section 108. The first section 108 can be closer to the proximal end 102 than the distal end 104. The first section 108 can have a first cross-sectional shape. The first section 108 can extend distally from the fastener section 106. The bone tie 100 can include a second section 110. The second section 110 can be closer to the proximal end 102 than the distal end 104. The second section 110 can have a second cross-sectional shape. The second section 110 can extend distally from the first section 108. The bone tie 100 can include a third section 112. The third section 112 can be closer to the distal end 104 than the proximal end 102. The third section 112 can have a third cross-sectional shape. The third section 112 can extend distally from the second section 110.

The bone tie 100 can include a neck section 114. The neck section 114 can be closer to the distal end 104 than the proximal end 102. The neck section 114 can taper from the third section 112 toward the distal end 104. The neck section 114 can extend distally from the third section 112. The neck section 114 can facilitate manipulation of the distal portion of the bone tie 100 by the bone tie inserter. The neck section 114 can be shaped to interface with the bone tie inserter. The neck section 114 can be shaped to form a mechanical interfit or coupling.

The bone tie 100 can include a head section 116. The head section 116 can be located at or near the distal end 104. The neck section 114 can taper toward the head section 116. The head section 116 can extend distally from the neck section 114. The head section 116 can facilitate manipulation of the distal portion of the bone tie 100 by the bone tie inserter. The head section 116 can be shaped to be grasped or cupped by the bone tie inserter. The head section 116 can be shaped to pivot and/or rotate relative to the bone tie inserter.

FIG. 3 is a perspective back view of the bone tie 100. The bone tie 100 can have a smooth surface along the first section 108, the second section 110, and the third section 112. The bone tie 100 can have a continuous surface along the first section 108, the second section 110, and the third section 112.

FIG. 4 illustrates a perspective view of a proximal portion of the bone tie 100. The bone tie can include the proximal end 102, the fastener section 106, first section 108, and the second section 110.

The fastener section 106 can include a lumen 118. The lumen 118 can be oriented perpendicular to a longitudinal axis 150 of the bone tie 100. The bone tie 100 can include a ratchet 122 disposed within the lumen 118. The ratchet 122 is configured to deflect to allow one or more gears to travel through the lumen 118 in one direction, but limit or prevent travel in another direction. The fastener section 106 can form an enlarged end of the bone tie 100. The fastener section 106 can be generally rectangular or cuboid. The fastener section 106 can have a width larger than the first section 108. The fastener section 106 can have a thickness larger than the first section 108. The fastener section 106 can include rounded edges or corners. The fastener section 106 can have any shape to accommodate the ratchet 122 disposed therewithin. The fastener section 106 can have any shape to accommodate any fastener mechanism described herein.

The first section 108 can have the first cross-sectional shape. The first cross-sectional shape can be generally rectangular or cuboid. The first cross-sectional shape can have rounded edges or corners. The first section 108 can include a width and a thickness. The first section 108 can include a groove 124. The groove 124 can reduce the thickness of the first section 108. The groove 124 can taper from the fastener section 106. The groove 124 can taper to the second section 110.

The second section 110 can have the second cross-sectional shape. The second cross-sectional shape can be generally rectangular or cuboid. The second cross-sectional shape can have rounded edges or corners. The second section 110 can include a groove 126. The groove 124 of the first section 108 can extend to the groove 126 of the second section 110. The second section 110 can include one or more gears 128. The gears 128 can be ramped surfaces. The gears 128 can form a rack. The gears 128 can be wedge surfaces. The gears 128 can be inclined upward toward the proximal end 102. The gears 128 can be inclined downward toward the distal end 104. The gears 128 can be disposed within the groove 126 of the second section 110. The first section 108 and the second section 110 can include a constant width. The first section 108 and the second section 110 can include a constant thickness. The first section 108 and the second section 110 can include a constant thickness measured along the edges of the first section 108 and the second section 110.

FIG. 5 illustrates a perspective view of a distal portion of the bone tie 100. The bone tie can include the second section 110, the third section 112, the neck section 114, the head section 116, and the distal end 104.

The third section 112 can have a third cross-sectional shape. The third cross-sectional shape can be generally rectangular or cuboid. The third cross-sectional shape can have rounded edges or corners. In some embodiments, the first cross-sectional shape and the third cross-sectional shape are the same or similar. The third section 112 can include a width and a thickness. The third section 112 can include a groove 130. The groove 130 can reduce the thickness of the third section 112. The groove 130 can taper from the second section 110. The groove 130 can taper to the neck section 114.

Two or more of the first section 108, the second section 110, and the third section 112 can include a constant width. Two or more of the first section 108, the second section 110, and the third section 112 can include a constant thickness. Two or more of the first section 108, the second section 110, and the third section 112 can include a constant thickness measured along the edges of the respective sections. The bone tie 100 can have a constant width along a substantial portion of the length. The bone tie 100 can have a constant thickness along a substantial portion of the length.

FIG. 6 illustrates an enlarged view of the distal portion of the bone tie 100. The bone tie 100 can include the neck section 114. The neck section 114 tapers along the width. The neck section 114 tapers from a larger width near the third section 112 to a smaller width near the head section 116. The neck section 114 can include a groove 132. The groove 132 can reduce the thickness of the neck section 114. The groove 132 of the neck section 114 can extend from the groove 130 of the third section 112.

The neck section 114 can lie in a plane along the longitudinal axis 150 of the bone tie 100 or the neck section 114 can include a curve 134. The curve 134 can have a constant radius of curvature. Two or more of the first section 108, the second section 110, and the third section 112 can be planar. The bone tie 100 can lie in a plane along a substantial portion of the length. The curve 134 can extend from the plane of the bone tie. The curve 134 can extend upward from the grooves 124, 126, 130, 132 of the bone tie 100. The curve 134 can extend upward from the gears 128 of the second section 110. The curve 134 can extend away from the longitudinal axis 150 of the bone tie 100.

The bone tie 100 can include the head section 116. The head section 116 can include a head 136. The head 136 can be rounded. The head 136 can be spherical. The head 136 can extend to the distal end 104 of the bone tie 100. The head section 116 can include a flange 138. The flange 138 can be positioned on the head 136. The flange 138 can be a rounded bill that extends from the head 136. The flange 138 can include a first tapered surface 140 and a second tapered surface 142. The first tapered surface 140 and the second tapered surface 142 can have different slopes. The second tapered surface 142 can form a ledge by which the head section 116 or head 136 can be grasped. The first tapered surface 140 and the second tapered surface 142 extend to the neck section 114.

The bone tie 100 can include a marker 144. The marker 144 can facilitate visualization of the bone tie 100, or a portion thereof. In the illustrated embodiment, the head 136 can include the marker 144. The head 136 can include a bore 146. The bore 146 can extend from an edge of the head 136 inward toward or past the center of the head 136. The marker 144 can be disposed within the bore 146. The marker 144 can be a radiopaque marker. The marker 144 can be formed of a metal or other radiopaque material. The marker 144 can identify the distal end 104 of the bone tie 100. In some embodiments, the bone tie 100 comprises a non-radiopaque material. In some embodiments, one or more radiopaque markers may be embedded in or on the bone tie 100 to assist in placement or monitoring of the bone tie 100 under radiographic visualization.

The bone tie 100 can be a flexible fastening band. The bone tie 100 can include the proximal end portion 102 and the distal end portion 104. In some embodiments, the head section 116 can be removed. The neck section 114 can be advanced through the lumen 118. When the neck section 114 is advanced, the ratchet 122 can extend into the groove 132. The third section 112 can be advanced through the lumen 118. When the third section 112 is advanced, the ratchet 122 can extend into the groove 130. The second section 110 can be advanced through the lumen 118. When the second section 110 is advanced, the ratchet 122 can extend into the groove 126. The ratchet 122 can engage the gears 128. The ratchet 122 can allow the second section 110 to travel through the lumen 118 in one direction, but limit travel through the lumen 118 in the opposite direction.

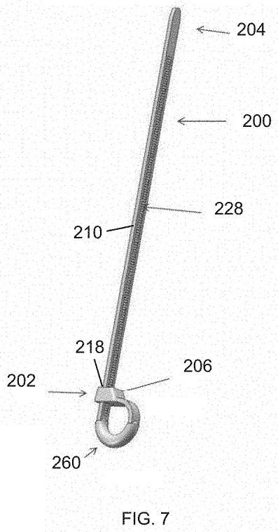

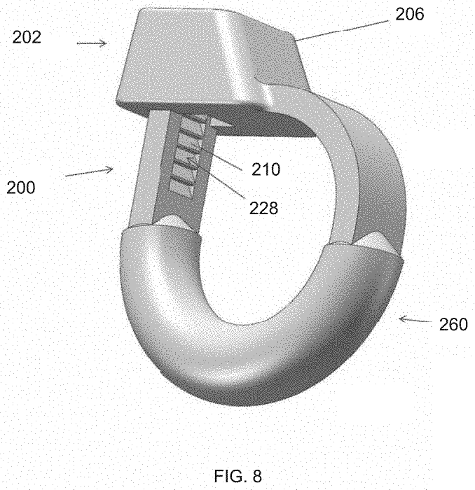

FIGS. 7 and 8 are perspective views of a bone tie 200. The bone tie 200 can include any of the features of the bone tie 100. The bone tie 200 can comprise a proximal end 202 and a distal end 204. The distal end 204 can be the leading end of the bone tie 200. The bone tie 200 can include one or more sections along the length of the bone tie 200. The sections can have a different cross-sectional shape than an adjacent section of the bone tie 200. The sections can have a different length than an adjacent section of the bone tie 200. The sections can have a different function than an adjacent section of the bone tie 200. The bone tie 200 can include a fastener section 206. The fastener section 206 can be located at or near the proximal end 202. The fastener section 206 can include a mechanism configured to form a loop. The fastener section 206 can include a mechanism that allows the bone tie 200 to be tightened in a single direction.

The distal end 204 of the bone tie 200 can have a tapered tip. The distal end 204 can facilitate passage through the fastener section 206. The fastener section 206 can include a lumen 218. The lumen 218 can be oriented through the fastener section. The bone tie 100 can include a ratchet disposed within the lumen 218. The ratchet is configured to engage one or more gears. The bone tie 200 can include at least a portion that includes one or more gears. The bone tie 200 can include at least a portion that does not include one or more gears. The bone tie 200 can have any cross-sectional shape. The section 210 can include one or more gears 228. The gears 228 can be ramped surfaces. The gears 228 can form a rack. The gears 228 can be wedge surfaces. The gears 228 can be inclined toward the proximal end 202. The gears 228 can be inclined toward the distal end 204. The fastener section 206 can include a ratchet 222 configured to engage the gears 228 formed in the bone tie 200. The fastener section 206 can allow the gears 228 to advance through the lumen 218 in only one direction.

The distal end 204 is configured to pass through a lumen formed through a vertebra. The distal end 204 is configured to pass through a lumen in the interbody device. The distal end 204 is configured to pass through the lumen 218 in the fastener section 206. The distal end 204 can be shaped to increase the ease of inserting the proximal end portion into the lumen 218 in the fastener section 206. The distal end 204 can be tapered, rounded, and/or angled to reduce at least a portion of a cross-sectional area of the distal end 204. The bone tie 200 can be monolithically formed.

The bone tie 200 can include one or more additional portions 260. The bone tie 200 can include a cylindrical portion. The bone tie 200 can include a cuboid portion. The bone tie 200 can include a portion having substantially the same cross-sectional dimension as the cross-sectional dimension of the lumen of a vertebra. The bone tie 200 can include a portion having substantially the same cross-sectional dimension as the cross-sectional dimension of a notch in an interbody device.

The bone tie 200 can have an excess length. The excess length can be removed, e.g., by cutting or breaking. The distal end 204 and a portion of the bone tie 200 is removed in FIG. 8. The bone tie 200 forms a loop. The bone tie 200 can be further tightened, but not loosened. The bone tie 200 can be removed by cutting or breaking the loop.

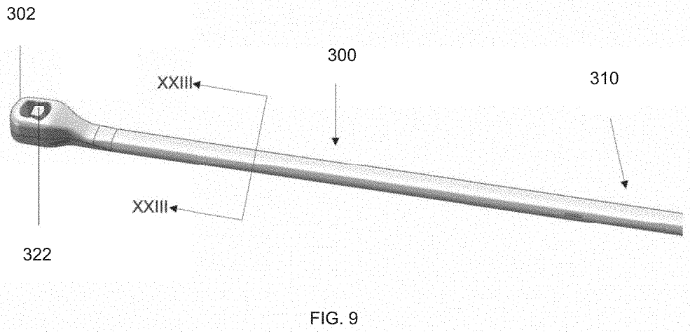

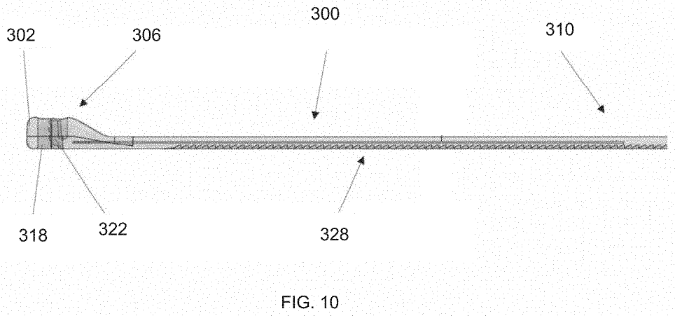

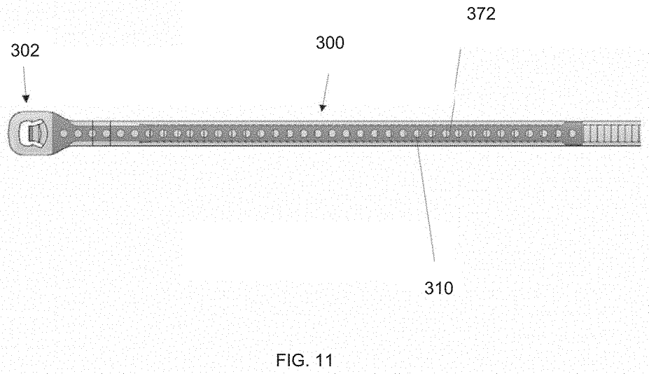

FIGS. 9-11 are views of a bone tie 300. FIG. 9 is a perspective view of the proximal portion of the bone tie 300. FIG. 10 is a side cross-sectional view of the proximal portion of the bone tie 300. FIG. 11 is a top view of the proximal portion of the bone tie 300.

The bone tie 300 can include any of the features of the bone tie 100 or bone tie 200. The bone tie 300 can comprise a proximal end 302 and a distal end (not shown). The distal end can have any features of the distal end 104, 204. The distal end can be the leading end of the bone tie 300. The bone tie 300 can include a fastener section 306. The fastener section 306 can include a mechanism that allows the bone tie 300 to be secured.

The fastener section 306 can include a lumen 318. The bone tie 300 can include a ratchet 322 disposed within the lumen 318. The ratchet 322 is configured to engage one or more gears 328 to allow tightening and securing of the bone tie 300. The bone tie 300 can include at least a portion that includes one or more gears 328. The section 310 can include one or more gears 328. The fastener section 306 can include a ratchet 322 configured to engage the gears 328. The fastener section 306 can allow the gears 328 to advance through lumen 318 in only one direction. The bone tie 300 can include a gear rack.

The bone tie 300 can include a reinforcement piece 372. The reinforcement piece 372 can include any of the materials described herein. The reinforcement piece 372 can include a material stronger than another portion of the bone tie 300. The reinforcement piece 372 can include a metal. The reinforcement piece 372 can include a polymer. The reinforcement piece 372 can be disposed within the bone tie 300. The reinforcement piece 372 can be disposed along the length of the bone tie 300. The reinforcement piece 372 can be closer to the proximal end 302 than the distal end. The reinforcement piece 372 can overlap with the one or more gears 328. The reinforcement piece 372 can be molded within the bone tie 300. The reinforcement piece 372 can have any shape. The reinforcement piece 372 can have any length. The reinforcement piece 372 can change the bending characteristics of the bone tie. The reinforcement piece 372 can change the torsion characteristics of the bone tie 300.

The bone tie 100, 200, 300 can have a width of 0.5 mm, 1 mm, 1.5 mm, 2 mm, 2.5 mm, 3 mm, 3.5 mm, 4 mm, or any range of the foregoing values. The width of the bone tie 100, 200, 300 can vary along the length of the bone tie 100, 200, 300. The bone tie 100, 200, 300 can have a thickness of 0.5 mm, 1 mm, 1.5 mm, 2 mm, 2.5 mm, 3 mm, 3.5 mm, 4 mm, or any range of the foregoing values. The thickness of the bone tie 100, 200, 300 can vary along the length of the bone tie 100, 200, 300. The bone tie 100, 200, 300 can have a length of 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, or any range of the foregoing values. For example, the bone tie 100, 200, 300 can have a length of 175 mm. In some embodiments, the second section 110, 210, 310 or the gears 128, 228, 328 can have a length of 5 mm, 10 mm, 15 mm, 20 mm, 25 mm, 30 mm, 35 mm, 40 mm, 45 mm, 50 mm, 60 mm, 65 mm, 70 mm, 75 mm, 80 mm, or any range of the foregoing values.

The bone tie 100, 200, 300 can be manufactured from any of a variety of materials known in the art, including but not limited to a polymer such as polyetheretherketone (PEEK), polyetherketoneketone (PEKK), polyethylene, fluoropolymer, hydrogel, or elastomer; a ceramic such as zirconia, alumina, or silicon nitride; a metal such as titanium, titanium alloy, cobalt chromium or stainless steel; or any combination of the materials described herein. The bone tie 100, 200, 300 can include any biocompatible material, e.g., stainless steel, titanium, PEEK, nylon, etc. In some embodiments, the bone tie 100, 200, 300 comprises at least two materials. The bone tie 100, 200, 300 can include a reinforcement piece disposed within the bone tie 100, 200, 300. By selecting a particular configuration and the one or more materials for the bone tie 100, 200, 300, the bone tie 100, 200, 300 can be designed to have the desired flexibility and resiliency.

In some embodiments, the bone tie 100, 200, 300 can form a unitary structure. The bone tie 100, 200, 300 can be integrally formed from the proximal end to the distal end. In some embodiments, the bone tie 100, 200, 300 can include one or more unitarily formed sections along the length of the bone tie 100, 200, 300. In some embodiments, the bone tie 100, 200, 300 can include one or more separately formed sections along the length of the bone tie 100, 200, 300. The bone tie 100, 200, 300 can be monolithically formed. The bone tie 100, 200, 300 can be formed of the same or similar material. The sections of the bone tie 100, 200, 300 can be formed of the same or similar construction. In some embodiments, the bone tie 100, 200, 300 is formed from an injection molding process. In some embodiments, the shape of the bone tie 100, 200, 300 can be determined based on the shape of an artificial lumen formed through a vertebra. In some embodiments, the shape of the bone tie 100, 200, 300 can be determined based on the shape of a notch or opening formed through the interbody device.

In some embodiments, the characteristic of the bone tie 100, 200, 300 can vary along the length of the bone tie 100, 200, 300. In some embodiments, the flexibility of the bone tie 100, 200, 300 varies along the length of the bone tie 100, 200, 300. In some embodiments, the torsional strength of the bone tie 100, 200, 300 varies along the length of the bone tie 100, 200, 300. In some embodiments, the resistance to deformation or elongation of the bone tie 100, 200, 300 varies along the length of the bone tie 100, 200, 300. In some embodiments, the characteristic of the bone tie 100, 200, 300 vary based, at least in part, on the shape of the various sections.

In some embodiments, the characteristic of the bone tie 100, 200, 300 vary based on the material of the various sections. In some embodiments, the characteristic of the bone tie 100, 200, 300 vary along the length based, at least in part, on a reinforcement piece. The reinforcement piece can be separately formed from or integrally formed with the bone tie 100, 200, 300. The reinforcement piece can comprise a different material or material property. The reinforcement piece can increase the strength of a section of the bone tie 100, 200, 300. The reinforcement piece can increase or decrease bending strength. The reinforcement piece can increase or decrease torsion strength. In some embodiments, the reinforcement piece is radiopaque. In some embodiments, the reinforcement piece is radiolucent.

3. Interbody Device

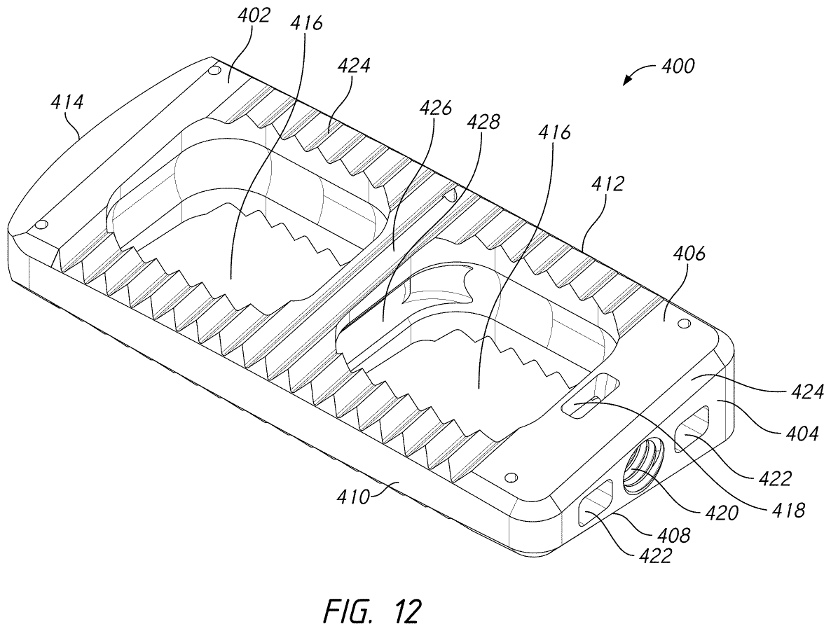

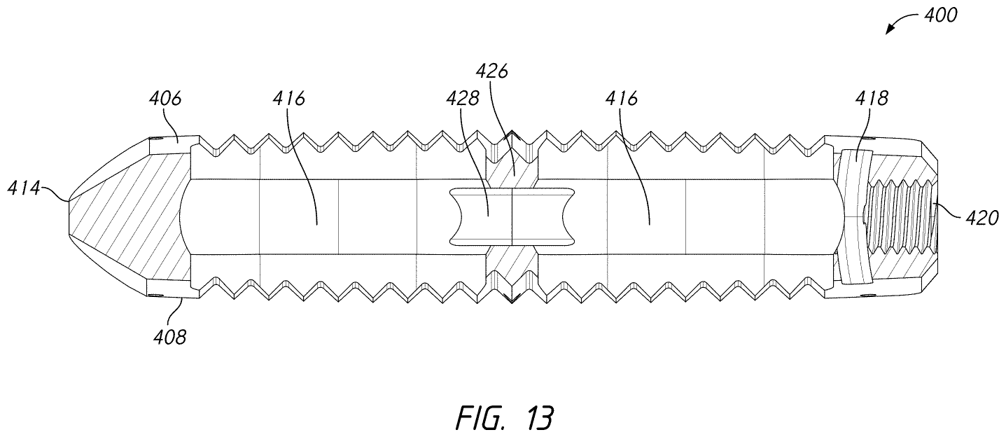

FIG. 12 is a view of an embodiment of an interbody device 400. FIG. 13 is a cross-sectional view of the interbody device 400. The interbody device 400 can comprise any structure configured to maintain a separation and resist compression between two vertebrae V1, V2. The interbody device 400 can have any of a variety of overall shapes, including but not limited to a rectangular box, a trapezoidal box, H-shaped, O-shaped, V-shaped, with or without one or more lumens within the spacing structure. The interbody device 400 comprises a body 402. The body 402 can be placed between vertebrae V1, V2. The body 402 can form the length of the interbody device 400. The body 402 can form the height of the interbody device 400. The body 402 can form the width of the interbody device 400. The interbody device 400 can include one or more rounded corners or edges. The interbody device 400 can include one or more sharp corners or edges. The interbody device 400 can include any feature described herein.

The interbody device 400 can be configured for insertion between any two vertebrae. The interbody device 400 can be configured for insertion between two cervical vertebrae. The interbody device 400 can be configured for insertion between two lumbar vertebrae. The interbody device 400 can be configured for insertion between two thoracic vertebrae. The interbody device 400 can be configured for any approach. The interbody device 400 can be configured for an anterior approach. The interbody device 400 can be configured for a posterior approach. The interbody device 400 can be configured for a lateral approach.

The body 402 can have a proximal end 404, a superior surface 406 and an inferior surface 408, and side surfaces 410, 412, and a distal end 414. In some methods of use, the distal end 414 can be the insertion end. In some embodiments, the proximal end 404 can be flat or flattened. In some embodiments, the proximal end 404 can include one or more rounded corners or edges. Each surface 404, 406, 408, 410, 412, 414 can be flat or flattened. Each surface 404, 406, 408, 410, 412, 414 can be curved or undulating. Each surface 404, 406, 408, 410, 412, 414 can be any combination of flattened or curved surfaces.

In some embodiments, the proximal end 404 can be flat or flattened. In some embodiments, the proximal end 404 can include one or more rounded corners or edges. The proximal end 404 can be a flat surface or generally flat surface. The proximal end 404 can be rounded toward the sides of the interbody device 400. The proximal end 404 can have a first radius of curvature extending from a first side surface. The proximal end 404 can have a second radius of curvature extending from a second side surface. The first radius of curvature and the second radius of curvature can be the same. The first radius of curvature and the second radius of curvature can be different.

The proximal end 404 can engage with an insertion tool as described herein. The proximal end 404 can have any shape or surface that facilitates engagement with the insertion tool. In some embodiments, the interbody device 400 can include an opening 420. The opening 420 can be circular or rounded. In some embodiments, the opening 420 is located at the proximal end 404. The opening 420 can be threaded to engage a threaded end of the insertion tool. The opening 420 can be centrally located. The opening 420 can be located at a neutral center of the interbody device 400. The opening 420 can be located along the longitudinal axis of the interbody device 400.

The interbody device 400 can include one or more features 422 to facilitate placement of the interbody device 400. In some embodiments, the one or more features 422 are located at the proximal end 404. The one or more features 422 can be rectangular. The one or more features 422 can be circular or rounded. The feature 422 can have at least one dimension that is smaller than the opening 420. The feature 422 can have a smaller height than the opening 420. The feature 422 can have a smaller width than the opening 420. The feature 422 can have a different shape than the opening 420. The one or more features 422 can be shaped to engage a complementary shaped end of the insertion tool. The one or more features 422 can be diametrically opposed relative to the opening 420. The one or more features 422 can be equally spaced relative to the opening 420. The opening 420 and the one or more features 422 can facilitate control of the interbody device 400. The opening 420 can prevent axial or translational movement between the interbody device 400 and the insertion tool. The one or more features 422 can prevent rotational movement between the interbody device 400 and the insertion tool. The proximal end 404 can include one or more undercuts 424. The top surface of the proximal end 404 can include the undercut 424. The lower surface of the proximal end 404 can include the undercut 424. The one or more undercuts 424 can facilitate engagement with the insertion tool. The one or more undercuts 424 can prevent rotational movement between the interbody device 400 and the insertion tool.

In some methods of use, the distal end 414 can be inserted into the space between adjacent vertebrae before another portion of the interbody device 400. In some embodiments, the distal end 414 forms a blunt or atraumatic shape. The distal end 414 can form a leading edge. The distal end 414 can include one or more sharp corners or edges. The distal end 414 can taper downward along an upper surface. The distal end 414 can taper upward along a lower surface. The distal end 414 can be rounded toward the sides of the interbody device 400. The distal end 414 can have a first radius of curvature extending from a first side surface. The distal end 414 can have a second radius of curvature extending from a second side surface. The first radius of curvature and the second radius of curvature can be the same. The first radius of curvature and the second radius of curvature can be different.

In some embodiments, the interbody device 400 can include a slight inclination toward one side of the interbody device 400. The interbody device 400 can have a lordosis angle. The interbody device 400 can have a lordosis angle to correspond to the natural orientation of the vertebral endplates. The lordosis angle can be zero. The lordosis angle can be an angle greater than zero. The lordosis angle can be an angle such as 0°, 1°, 2°, 3°, 4°, 5°, 6°, 7°, 8°, 9°, 10°, 11°, 12°, 13°, 14°, 15°, 16°, 17°, 18°, 19°, 20°, between 0° and 5°, between 0° and 6°, between 0° and 7°, between 3° and 9°, between 5° and 7°, between 6° and 12°, between 8° and 16°, between 10° and 14°, between 12° and 14°, between 16° and 20°, approximately 6°, approximately 12°, approximately 18°, or any range of two of the foregoing values. In some embodiments, the distal end 414 is tapered to one side by the lordosis angle as described herein. In some embodiments, the proximal end 404 is tapered to one side by the lordosis angle as described herein.

The superior surface 406 and the inferior surface 408 can be configured for facing the superior and inferior vertebral bodies adjacent to an implantation site. The relative configuration of the superior surface 406 and the inferior surface 408 can vary, depending upon the relative position desired between the two adjacent vertebrae, the anatomical shape of the vertebrae, ease of insertion of the implant and other factors. For example, if a neutral vertical alignment is desired between two vertebrae, the superior surface 406 and the inferior surface 408 can have generally parallel planar orientations. If a non-neutral alignment is desired, for instance to maintain a natural spinal curvature in the cervical region, the superior surface 406 and the inferior surface 408 can have a wedge-like relationship to allow fixation of the vertebrae in the desired non-neutral position. A non-neutral alignment with respect to the anterior-posterior direction can also be used to compensate for excessive lordosis or kyphosis in other portions of the vertebral column. The height of the body 402 at any section between the superior surface 406 and the inferior surface 408 can be further configured to accommodate degenerative changes or anatomical anomalies to provide fixation in the desired relative position.

The superior surface 406 can span between the distal end 414 and the proximal end 404, or a portion thereof. The superior surface 406 can form an upper surface of the interbody device 400, or a portion thereof. In some embodiments, the superior surface 406 can be solid. In some embodiments, the superior surface 406 can include one or more porous or network surfaces. The superior surface 406 can facilitate the load bearing capacity of the interbody device 400.

In some embodiments, the interbody device 400 can include one or more features to limit or reduce movement of the interbody device 400 between the vertebrae. The one or more features can allow movement in at least a first direction, such as an insertion direction. The one or more features can limit or reduce movement in at least a second direction, opposite the first direction. The one or more features can reduce the migration of the interbody device 400 in a direction opposite the insertion direction. The interbody device 400 can include a plurality of ridges 424. The plurality of ridges 424 can extend along a portion of the superior surface 406. The plurality of ridges 424 can extend along a proximal edge of the superior surface 406. The plurality of ridges 424 can extend along a distal edge of the superior surface 406. The plurality of ridges 424 can extend along one or both side edges of the superior surface 406.

The inferior surface 408 can span between the distal end 414 and the proximal end 404, or a portion thereof. The inferior surface 408 can form a lower surface of the interbody device 400, or a portion thereof. In some embodiments, the inferior surface 408 can be solid. In some embodiments, the inferior surface 408 can include one or more porous or network surfaces. The inferior surface 408 can facilitate the load bearing capacity of the interbody device 400.

The plurality of ridges 424 can extend along a portion of the inferior surface 408. The plurality of ridges 424 can extend along a proximal edge of the inferior surface 408. The plurality of ridges 424 can extend along a distal edge of the inferior surface 408. The plurality of ridges 424 can extend along one or both side edges of the inferior surface 408.

The side surfaces 410, 412 can be generally parallel or skewed. In some embodiments, the side surfaces 410, 412 taper toward the distal end 414. A tapered body 402 can facilitate insertion of the interbody device 400 into the intervertebral space. In some embodiments, the one or more side surfaces 410, 412 can flare outward. In some embodiments, the one or more side surfaces 410, 412 have both tapering and flaring portions. The interbody device 400 can be generally rectangular. The interbody device 400 can have a shape that mirrors the anatomy.

In some embodiments, the interbody device 400 can have a length measured between the distal end 414 and the proximal end 404. The interbody device 400 can include the side surfaces 410, 412. The side surfaces 410, 412 can include the same dimensions. The side surfaces 410, 412 can include the same features. The side surfaces 410, 412 can be the same or similar. The side surfaces 410, 412 can be identical. The side surfaces 410, 412 can include different dimensions. The side surfaces 410, 412 can include different heights due to the lordosis angle. The side surfaces 410, 412 can include different features.

The side surfaces 410, 412 can extend along the length of the interbody device 400, or a portion thereof. The side surfaces 410, 412 can be opposing side walls. In some embodiments, the side surfaces 410, 412 are parallel or generally parallel along at least a portion of the length of the interbody device 100. In some embodiments, the side surfaces 410, 412 are aligned or generally aligned along at least a portion of the length of the interbody device 400.

The interbody device 400 can have a length or depth. The side surfaces 410, 412 can define at least a portion of the length or depth of the interbody device 400. The interbody device 400 can define a range of lengths. The interbody device 400 can have a maximum length of 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, 60 mm, 61 mm, 62 mm, 63 mm, 64 mm, 65 mm, 66 mm, 67 mm, 68 mm, 69 mm, 70 mm, between 6 mm and 10 mm, between 10 mm and 15 mm, between 15 mm and 20 mm, between 23 mm and 27 mm, between 25 mm and 29 mm, between 27 mm and 31 mm, between 29 mm and 33 mm, between 40 mm and 60 mm, between 40 mm and 50 mm, between 50 mm and 60 mm, between 60 mm and 70 mm, or any range of two of the foregoing values. The side surfaces 410, 412 can have the same or similar length.

In some embodiments, the side surfaces 410, 412 can be spaced apart. The side surfaces 410, 412 can define the width of the interbody device 400. The width can vary along the length. The side surfaces 410, 412 can define a range of widths along at least a portion of the length of the interbody device 400. The maximum width as measured between the side surfaces 410, 412 can be 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm, 20.5 mm, 21 mm, 21.5 mm, 22 mm, 22.5 mm, 23 mm, 23.5 mm, 24 mm, 24.5 mm, 25 mm, 25.5 mm, 26 mm, 26.5 mm, 27 mm, 27.5 mm, 28 mm, 28.5 mm, 29 mm, 29.5 mm, 30 mm, 30.5 mm, 31 mm, 31.5 mm, 32 mm, 32.5 mm, 33 mm, 33.5 mm, 34 mm, 34.5 mm, 35 mm, 35.5 mm, 36 mm, 36.5 mm, 37 mm, 37.5 mm, 38 mm, 38.5 mm, 39 mm, 39.5 mm, 40 mm, 40.5 mm, 41 mm, 41.5 mm, 42 mm, 42.5 mm, 43 mm, 43.5 mm, 44 mm, 44.5 mm, 45 mm, between 10 mm and 15 mm, between 14 mm and 16 mm, between 16 mm and 18 mm, between 17 mm and 20 mm, between 18 mm and 22 mm, between 20 mm and 22 mm, between 28 mm and 32 mm, between 35 mm and 39 mm, between 39 mm and 43 mm, between 30 mm and 37 mm, between 30 mm and 41 mm, or any range of two of the foregoing values.

The side surfaces 410, 412 can extend along the height of the interbody device 100. The side surfaces 410, 412 can define the height of the interbody device 400. The height can vary based on the lordosis angle. The side surfaces 410, 412 can define a range of heights. The side surface 410 can have a maximum height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, or any range of two of the foregoing values. The side surface 412 can have a maximum height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, or any range of two of the foregoing values. The height of the side surface 410 can be less than the height of the side surface 412. The lordosis angle can taper downward from the side surfaces 412 toward the side surfaces 410. The height of the side surface 412 can be less than the height of the side surface 410. The lordosis angle can taper downward from the side surface 410 toward the side surface 412.

In some embodiments, the side surfaces 410, 412 can be flat or smooth. In some embodiments, the side surfaces 410, 412 can include one or more porous or network surfaces. In some embodiments, the side surfaces 410, 412 can be solid. In some embodiments, the side surfaces 410, 412 are linear. In some embodiments, the side surfaces 410, 412 are curved. The side surfaces 410, 412 410, 412 can be concave. The side surfaces 410, 412 can be convex.

The side surfaces 410, 412 can extend between the distal end 414 and the proximal end 404. In some embodiments, the side surfaces 410, 412 are separated by a similar width along a majority of the length of the side surfaces 410, 412. In some embodiments, the side surfaces 410, 412 are separated by a varying width along a majority of the length of the side surfaces 410, 412. In some embodiments, the side surfaces 410, 412 generally taper along a majority of the length of the side surfaces 410, 412. In some embodiments, the side surfaces 410, 412 are generally at an angle to each other along a majority of the length of the side surfaces 410, 412. In some embodiments, the side surfaces 410, 412 taper to the distal end 414. In some embodiments, the side surfaces 410, 412 have a smaller width near the distal end 414 than the proximal end 404. In some embodiments, the side surfaces 410, 412 are straight or generally straight along a majority of the length of the side surfaces 410, 412. In some embodiments, the side surfaces 410, 412 are slightly curved along a majority of the length of the side surfaces 410, 412. The side surfaces 410, 412 can bow outward. The side surfaces 410, 412 can bow inward.

The interbody device 400 can include one or more windows 416. The one or more windows 416 can create a passageway in a vertical direction. The one or more windows 416 can allow ingrowth as described herein. The one or more windows 416 promote fusion through the one or more windows 416. The one or more windows 416 can extend along the height of the interbody device 400. The one or more windows 416 can be diametrically opposed. The one or more windows 416 can form a through lumen through the interbody device 400. The one or more windows 416 can allow fusion in the vertical direction. The one or more windows 416 can allow fusion between adjacent vertebral endplates. The one or more windows 416 can form a passageway between adjacent vertebrae. These windows 416 can allow bony growth into the interbody device 400. The windows 416 can be filled with graft materials. The graft material can be an autograft, allograft, xenograft or synthetic material. Synthetic graft material can be ceramic-based, silicon-based or calcium-based. The graft material can also include osteoinductive factors to promote bone ingrowth. The interbody device 400 can include two windows 416. The interbody device 400 can include a graft chamber. In some embodiments, the one or more windows 416 extend from the superior surface 406. In some embodiments, the one or more windows 416 extend from the inferior surface 408. In some embodiments, the one or more windows 416 extend vertically from the superior surface 406 to the inferior surface 408. In some embodiments, the one or more windows 416 extend from the side surface 410. In some embodiments, the one or more windows 416 extend from the side surface 412. In some embodiments, the one or more windows 416 extend laterally from the side surface 410 to the side surface 412. The interbody device 400 can include one or more supports 426. The one or more supports 426 can separate the one or more windows 416. The one or more supports 426 can allow passage of graft material between the one or more windows 416. The one or more supports 426 can include a horizontal lumen 428. The horizontal lumen 428 can be in communication with the one or more windows 416. The one or more supports 426 can be separated by the horizontal lumen 428. The one or more supports 426 can be open between the upper surface 406 and the lower surface 408. The one or more supports 426 can extend along the superior surface 406. The one or more supports 426 can extend along the inferior surface 408.

The interbody device 400 can have one or more openings or notches 418. The notch 418 can extend from the superior surface 406. The notch 418 can extend to the window 416. The notch 418 can extend from the superior surface 406 to the window 416. The notch 418 can extend from the inferior surface 408. The notch 418 can extend to the window 416. The notch 418 can extend from the inferior surface 408 to the window 416. The notch 418 can form a lumen. The notch 418 can form a passageway. The notch 418 can form an arc. The notch 418 can be continuous. The notch 418 can be discontinuous.

FIGS. 12 and 13 illustrate an embodiment of the notch 418. The notch 418 is in communication with the window 416. The notch 418 is through the interbody device 400. The notch 418 is open toward the window 416. The interbody device 400 can include the notch 418 to allow a drill to move through interbody device 400. The notch 418 can match the shape of the arc formed by the drill. The interbody device 400 can include the notch 418 to allow passage of the bone tie 100, 200, 300. The notch 418 can match the shape of the bone tie 100, 200, 300. The notch 418 can be rectangular, polygonal, or square. The notch 418 can be circular, rounded, elliptical, or oval. The notch 418 can be elongated. The notch 418 can have a cross-sectional shape that matches the cross-sectional shape of the bone tie 100, 200, 300. The notch 418 can have a cross-sectional shape that is different from the cross-sectional shape of the bone tie 100, 200, 300. The notch 418 can accommodate the bone tie 100, 200, 300. The notch 418 can receive a single bone tie 100, 200, 300. In some embodiments, the notch 418 can receive one or more bone ties 100, 200, 300.

The notch 418 can be in contact with graft material for facilitating fusion. The notch 418 can be in communication with the window 416. The notch 418 can be open along the length. The notch 418 can be closed near the superior surface 406. The notch 418 can be closed near the inferior surface 408. The notch 418 can be open in between the superior surface 406 and the inferior surface 408. The notch 418 can be open near a midpoint of the notch 418. The notch 418 can be open along a portion of the window 416. The bone tie 100, 200, 300 can be in contact with graft material for facilitating fusion when inserted into the notch 418. The bone tie 100, 200, 300 disposed within the notch 418 can be in communication with the window 416. The window 416 can be packed with material before insertion of the bone tie 100, 200, 300 in the notch 418. The window 416 can be packed with material after insertion of the bone tie 100, 200, 300 in the notch 418. The window 416 can be packed with material vertically from either the opening in the superior surface 406 or the inferior surface 408. The window 416 can be packed with material laterally through the opening 420. The window 416 can be packed with material laterally through the one or more features 422.

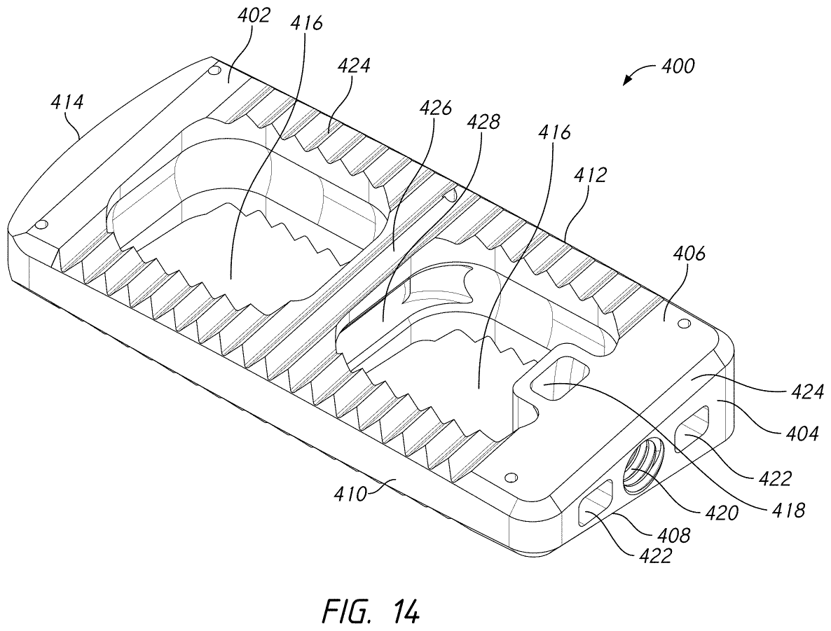

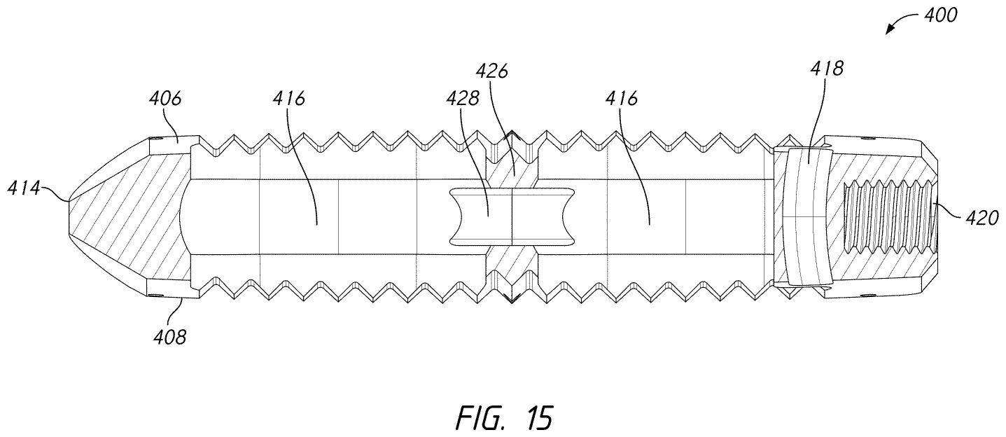

FIGS. 14 and 15 illustrate another embodiment of the notch 418. The notch 418 is not in communication with the window 416. The notch 418 is closed toward the window 416. The notch 418 is through the interbody device 400. The notch 418 is continuous from the superior surface 406 to the inferior surface 408. The notch 418 is closed toward the window 416. The notch 418 can form a channel through the interbody device 400 to allow the drill to move through interbody device 400. The notch 418 can form a channel with clearance for a curved drill. The notch 418 can form a channel for the bone tie 100, 200, 300. The notch 418 can surround the bone tie 100, 200, 300. The notch 418 can accommodate the bone tie 100, 200, 300. The bone tie 100, 200, 300 can be separated from the graft material within the window 416. The notch 418 can prevent or limit graft material from entering the passageway. The notch 418 can keep the passageway clear for the drill and the bone tie 100, 200, 300. The bone tie 100, 200, 300 disposed within the notch 418 can be separated from the window 416. The window 416 can be packed with material before insertion of the bone tie 100, 200, 300 in the notch 418. The window 416 can be packed with material vertically from either the opening in the superior surface 406 or the inferior surface 408. The window 416 can be separated from the opening 420. The window 416 can be separated from the one or more features 422. The notch 418 can receive a single bone tie 100, 200, 300. In some embodiments, the notch 418 can receive one or more bone ties 100, 200, 300.

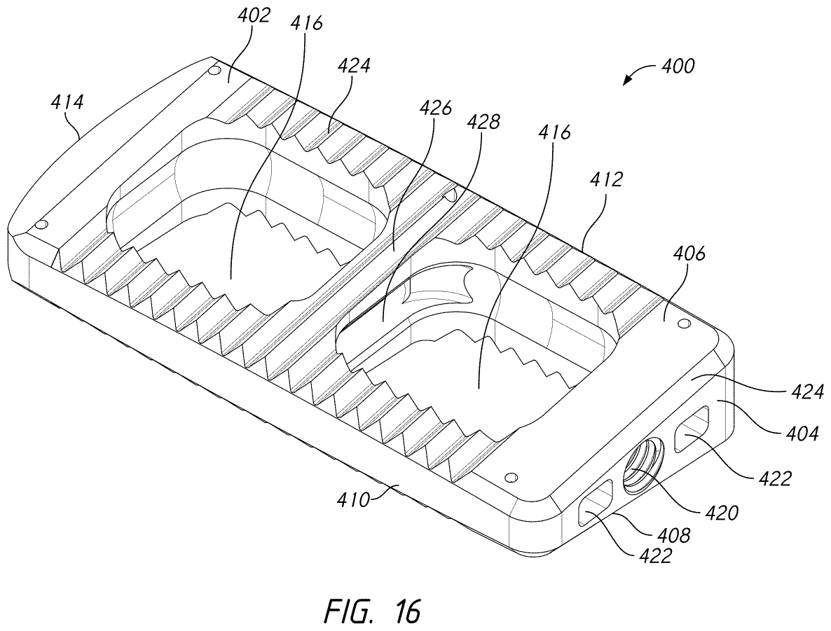

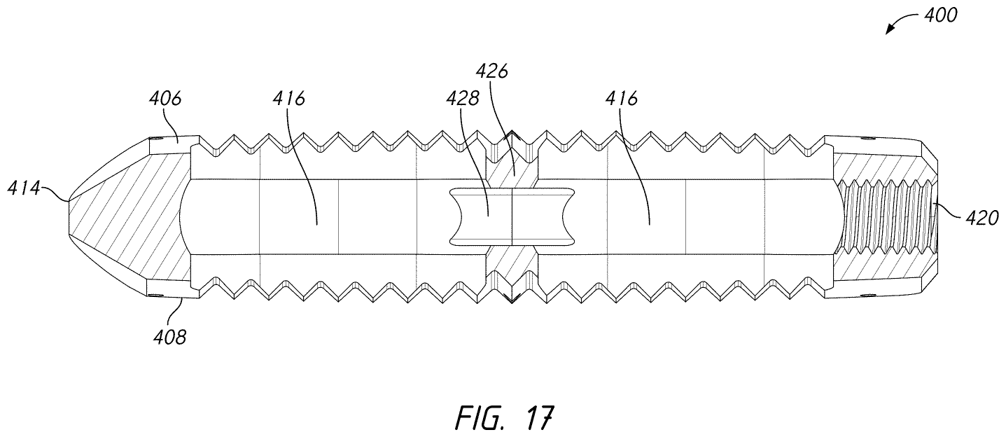

FIGS. 16 and 17 illustrate another embodiment of the interbody device 400. The interbody device 400 can omit the notch 418. The interbody device 400 can include the graft window 416. The interbody device 400 can include two or more graft windows 416. The graft window 416 can allow a drill to move through interbody device 400. The drill can pass through a proximal window 416. The drill can pass through the window 416 near the proximal end 404. The drill can pass through a distal window 416. The drill can pass through the window 416 near the distal end 414. The drill can pass through the window 416 along the length of the interbody device 400. The graft window 416 can allow the bone tie 100, 200, 300 to move through the interbody device 400. The graft window 416 can accommodate the bone tie 100, 200, 300. The bone tie 100, 200, 300 can be in contact with graft material for facilitating fusion when the bone tie 100, 200, 300 is inserted in the window 416. The window 416 can be packed with material before insertion of the bone tie 100, 200, 300. The window 416 can be packed with material vertically from either the opening in the superior surface 406 or the inferior surface 408. The window 416 can be packed with material laterally through the opening 420. The window 416 can be packed with material laterally through the one or more features 422. The window 416 can receive a single bone tie 100, 200, 300. In some embodiments, the window 416 can receive one or more bone ties 100, 200, 300.

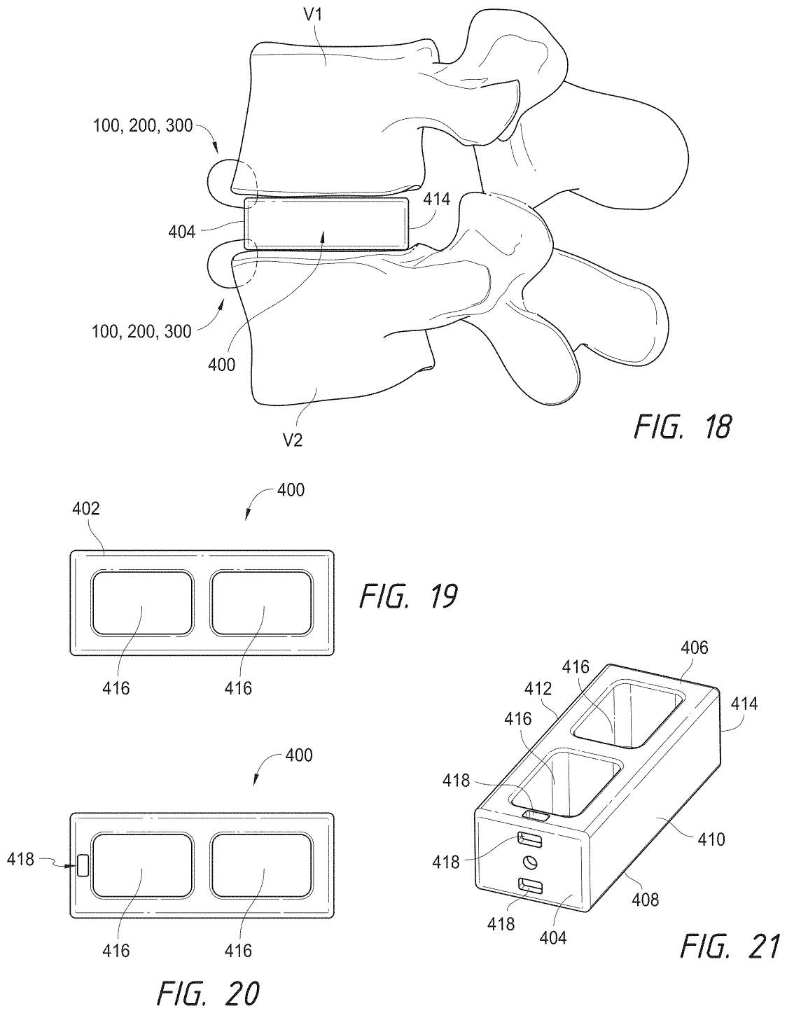

FIG. 18 is a side view of an embodiment of an interbody device 400 and the bone tie 100, 200, 300. The interbody device 400 can have any feature or combination of features described herein. The interbody device 400 can be configured to engage a single bone tie 100, 200, 300. The interbody device 400 can be configured to engage two bone ties 100, 200, 300. In some embodiments, each bone tie 100, 200, 300 can extend through a separate notch 418. In some embodiments, two or more bone ties 100, 200, 300 extend through a single notch 418. In some embodiments, two or more bone ties 100, 200, 300 extend through a single window 416. In some embodiments, one or more bone ties 100, 200, 300 extend through a notch 418 and one or more bone ties 100, 200, 300 extend through a window 416. FIG. 18 illustrates an embodiment of the interbody device 400 engaging two bone ties 100, 200, 300. FIG. 19 is a view of an embodiment of the interbody device 400. FIG. 20 is a view of an embodiment of the interbody device 400. FIG. 21 is a perspective view of an embodiment of the interbody device 400. FIGS. 18-20 illustrate views of the one or more windows 416 and the one or more notches 418.

The interbody device 400 can include one or more notches 418. The interbody device 400 can include two notches 418. The first notch 418 can extend from the proximal end 404. The first notch 418 can extend from the proximal end 404 to the superior surface 406. The first notch 418 can form a linear path from the proximal end 404 to the superior surface 406. The first notch 418 can form a non-linear path from the proximal end 404 to the superior surface 406. The first notch 418 can form a curved path from the proximal end 404 to the superior surface 406. The second notch 418 can extend from the proximal end 404 to the inferior surface 408. The second notch 418 can form a linear path from the proximal end 404 to the inferior surface 408. The second notch 418 can form a non-linear path from the proximal end 404 to the inferior surface 408. The second notch 418 can form a curved path from the proximal end 404 to the inferior surface 408. The notches 418 can follow the arc of a drill. FIG. 21 illustrates the first notch 418 and the second notch 418.

Other configurations are contemplated. The notch 418 can extend from the proximal end 404 to the side surface 410. The notch 418 can form a linear path from the proximal end 404 to the side surface 410. The notch 418 can form a non-linear path from the proximal end 404 to the side surface 410. The notch 418 can form a curved path from the proximal end 404 to the side surface 410. The notch 418 can extend from the proximal end 404 to the side surface 412. The notch 418 can form a linear path from the proximal end 404 to the side surface 412. The notch 418 can form a non-linear path from the proximal end 404 to the side surface 412. The notch 418 can form a curved path from the proximal end 404 to the side surface 412. The notch 418 can extend from the proximal end 404 to the distal end 414. The notch 418 can form a linear path from the proximal end 404 to the distal end 414. The notch 418 can form a non-linear path from the proximal end 404 to the distal end 414. The notch 418 can form a curved path from the proximal end 404 to the distal end 414. The notch 418 can extend from the proximal end 404 to the window 416. The notch 418 can form a linear path from the proximal end 404 to the window 416. The notch 418 can form a non-linear path from the proximal end 404 to the window 416. The notch 418 can form a curved path from the proximal end 404 to the window 416.

The notch 418 can extend from the superior surface 406 to the window 416. The notch 418 can form a linear path from the superior surface 406 to the window 416. The notch 418 can form a non-linear path from the superior surface 406 to the window 416. The notch 418 can form a curved path from the superior surface 406 to the window 416. The notch 418 can extend from the inferior surface 408 to the window 416. The notch 418 can form a linear path from the inferior surface 408 to the window 416. The notch 418 can form a non-linear path from the inferior surface 408 to the window 416. The notch 418 can form a curved path from the inferior surface 408 to the window 416. The notch 418 can extend from the superior surface 406 to the inferior surface 408. The notch 418 can form a linear path from the superior surface 406 to the inferior surface 408. The notch 418 can form a non-linear path from the superior surface 406 to the inferior surface 408. The notch 418 can form a curved path from the superior surface 406 to the inferior surface 408.

The notch 418 can form a closed channel. The notch 418 can form an open channel. The notch 418 can be in communication with the window 416. The notch 418 and the window 416 can be walled off. The notch 418 can be separated from the window 416. The notch 418 can be partially open. The notch 418 can be open to the window 416. The notch 418 can be closed to the window 416. The notch 418 can be open to the opening 420. The notch 418 can be closed to the opening 420. The notch 418 can contact graft material. The notch 418 can be separated from graft material.

In some embodiments, the notch 418 can be omitted. The bone tie 100, 200, 300 can extend through the window 416. FIG. 16 illustrates an embodiment without notch 418. The bone tie 100, 200, 300 can extend through the first window 416. The bone tie 100, 200, 300 can extend through the second window 416. The window 416 can form an enlarged passageway for the bone tie 100, 200, 300. The window 416 can form a larger target for passage of the drill. In some embodiments, the window 416 can be omitted. In some embodiments, the window 416 closest to the proximal end 404 can be omitted. In some embodiments, the window 416 closest to the distal end 414 can be omitted. The bone tie 100, 200, 300 can extend through the notch 418. The notch 418 can be separated from one or more windows 416.

FIGS. 20 and 21 illustrate various embodiments of the notch 418. The interbody device 400 can include one notch 418. The interbody device 400 can include two or more notches 418. The two or more notches 418 can be symmetrical. The two or more notches 418 can be identical. The two or more notches 418 can be mirror images. The two or more notches 418 can be non-symmetrical. The two or more notches 418 can be different. The two or more notches 418 can have different cross-sectional shapes. The two or more notches 418 can received different shapes of bone ties. The two or more notches 418 can have differently shaped channels. The interbody device 400 can omit the notch 418. The bone tie 100, 200, 300 can loop through the window 416. In FIG. 16, the bone tie 100, 200, 300 can loop through the window 416. Two or more bone ties 100, 200, 300 can loop through a single window 416. One or more bone ties 100, 200, 300 can loop through the window 416 closest to the proximal end 404. One or more bone ties 100, 200, 300 can loop through the window 416 closest to the distal end 414. One or more bone ties 100, 200, 300 can extend generally vertically through the interbody device 400. One or more bone ties 100, 200, 300 can extend through the interbody device 400 along an arc. One or more bone ties 100, 200, 300 can extend through the interbody device 400 along a linear path. One or more bone ties 100, 200, 300 can extend through the interbody device 400 along a non-linear path. One or more bone ties 100, 200, 300 can extend through the interbody device 400 through the notch 418. One or more bone ties 100, 200, 300 can extend through the interbody device 400 through a portion of a notch 418 and a portion of the window 416. One or more bone ties 100, 200, 300 can extend through the interbody device 400 through a superior portion of the notch 418, a portion of the window 416, and an inferior portion of the notch 418.

Other embodiments are contemplated. The notch 418 can extend from the distal end 414. The notch 418 can extend from the distal end 414 to the superior surface 406. The notch 418 can extend from the distal end 414 to the inferior surface 408. The notch 418 can extend from the distal end 414 to the side surface 410. The notch 418 can extend from the distal end 414 to the side surface 412. The notch 418 can extend from the distal end 414 to the window 416.

The notch 418 can extend from the superior surface 406. The notch 418 can extend from the superior surface 406 to the inferior surface 408. The notch 418 can extend from the superior surface 406 to the window 416. The notch 418 can extend from the side surface 410. The notch 418 can extend from the side surface 412. The notch 418 can extend from the side surface 410 to the side surface 412. The notch 418 can extend from the side surface 410 to the window 416. The notch 418 can extend from the side surface 412 to the window 416.

In some embodiments, the notch 418 is configured to accept the bone tie 100, 200, 300. The notch 418 is configured to accept the bone tie 100, 200, 300 for anchoring the interbody device 400 to the vertebra. The notch 418 can be slanted or orientated such that the opening is directed towards the superior vertebral body. The notch 418 can be slanted or orientated such that the opening is directed towards the inferior vertebral body. The notch 418 can be slanted or orientated such that the opening extends toward the posterior end 414. The notch 418 can be orientated such that the notch 418 extends generally vertically. The notch 418 can be linear. The notch 418 can be non-linear. The notch 418 can be straight. The notch 418 can be curved. The notch 418 can include a linear segment and a non-linear segment. The notch 418 can be open. The notch 418 can be closed. In some embodiments, the notch 418 can form a percentage of the surface area of superior surface 406 such as at least 5%, 10%, 15%, 20%, 25%, 30%, 35%, 40%, 45%, 50%, 55%, 60%, 65%, 70%, 75% or any range of two of the foregoing values. In some embodiments, the notch 418 can form a percentage of the surface area of the inferior surface 408 such as at least 5%, 10%, 15%, 20%, 25%, 30%, 35%, 40%, 45%, 50%, 55%, 60%, 65%, 70%, 75% or any range of two of the foregoing values.

In some embodiments, the window 416 is configured to accept the bone tie 100, 200, 300. The window 416 can be configured to accept the bone tie 100, 200, 300 for anchoring the interbody device 400 to the vertebra. The window 416 can be orientated such that the window 416 is open towards the superior vertebral body. The window 416 can be orientated such that the window 416 is open towards the inferior vertebral body. The window 416 can be orientated such that the window 416 extends generally vertically. The window 416 can be linear. The window 416 can be non-linear. The window 416 can be straight. The window 416 can be curved. The window 416 can be open on both the superior surface 406 and the inferior surface 408. The window 416 can form a partially enclosed cavity for graft material. The window 416 can be less than the entire length of the interbody device 400. In some embodiments, the window 416 can form a percentage of the surface area of the superior surface 406 such as at least 5%, 10%, 15%, 20%, 25%, 30%, 35%, 40%, 45%, 50%, 55%, 60%, 65%, 70%, 75% or any range of two of the foregoing values. In some embodiments, the window 416 can form a percentage of the surface area of the inferior surface 408 such as at least 5%, 10%, 15%, 20%, 25%, 30%, 35%, 40%, 45%, 50%, 55%, 60%, 65%, 70%, 75% or any range of two of the foregoing values.

The notch 418 can be configured to have the same or similar cross-section as the bone tie 100, 200, 300. The distal end of the bone tie 100, 200, 300 may be guided toward a lumen in a vertebra as the bone tie 100, 200, 300 is passed thorough the notch 418. The lumen can be formed by a drill, as described herein. The drill can determine the trajectory of the bone tie 100, 200, 300. The lumen in a vertebra can determine the trajectory of the bone tie 100, 200, 300. The notch 418 can determine the trajectory of the bone tie 100, 200, 300. The window 416 can determine the trajectory of the bone tie 100, 200, 300. The notch 418 can align with an artificial lumen created in the vertebral body. The guidance by the notch 418 advantageously prevents the bone tie 100, 200, 300 from being deflected away from the vertebral body as it is inserted into the bone. The notch 418 may prevent rotation of the bone tie 100, 200, 300 relative to the notch 418. The notch 418 can have a non-circular cross-sectional dimension. The notch 418 can have a rectangular cross-sectional dimension. In some embodiments, the notch 418 can receive the bone tie 100, 200, 300 in a single orientation. In some embodiments, the notch 418 can receive the bone tie 100, 200, 300 in two orientations.

One or more surfaces of the interbody device 400 can also have surface projections, indentations, or holes or pores that can further alter the characteristics of the interbody device 400. In some embodiments, angled projections, barbs, teeth or ramped surfaces can incline outwardly from one or more surfaces. In some embodiments, the ramped surfaces can be provided on one or more surfaces that facilitates insertion of the interbody device 400 in one direction but resists movement in the opposite direction. The ramped surfaces can be advantageous in reducing the migration of the interbody device 400 out of the intervertebral space. The ramped surfaces can maintain the position of the interbody device 400 during initial placement between vertebral bodies. The ramped surfaces can also reduce the forces acting upon the bone ties 100, 200, 300. The ramped surfaces can be provided on the superior surface 406. The ramped surfaces can be provided on the inferior surface 408. The interbody device 400 can include indentations, holes or pores for allowing bony ingrowth or filling with bony matrix or graft materials. These holes can be utilized with other surface features to further enhance insertion and stabilization of the interbody device 400.

In some embodiments, the interbody device 400 can have a height of about 4 mm to about 24 mm, or preferably about 4 mm to about 12 mm. In some embodiments, the interbody device 400 can have a height of about 6 mm to about 9 mm. In some embodiments, the interbody device 400 can have a length as measured from the proximal end 404 to the distal end 414 of about 40 mm to about 60 mm. In some embodiments, the length of the interbody device 400 can be about 5 mm to about 25 mm. In some embodiments, the length of the interbody device 400 can be about 10 mm to about 15 mm. The width of the interbody device 400 can be generally about 5 mm to about 25 mm, and in some situations, about 10 mm to about 15 mm.

The interbody device 400 can include, be made of, treated, coated, filled, used in combination with, or contain artificial or naturally occurring materials suitable for implantation in the human spine. These materials can include any source of osteogenesis, bone growth-promoting materials, bone derived substances, bone morphogenetic proteins, hydroxyapatite, genes coding for the production of bone, and bone including, but not limited to, cortical bone. The interbody device 400 can also be formed of material such as metal including, but not limited to, titanium and its alloys, surgical grade plastics, plastic composites, ceramics, or other materials suitable for use as a spinal fusion implant. In some embodiments, the interbody device 400 comprises a polyaryl polymer, including but not limited to PEK, PEEK, PEKK, PEKEKK or a blend thereof. The interbody device 400 can include any material described herein. In some embodiments, the interbody device 400 can comprise a radiolucent material, a radio-opaque material, or a combination thereof. The interbody device 400 can be partially or completely radiolucent, which can be advantageous when evaluating the effect of the implant post-implantation. Many existing spinal fixation plates and/or spacers obscure visualization of the vertebrae, which can complicate post-operative treatment, diagnosis and prognosis of the patient's condition. The interbody device 400 can include at least in part materials that are bioabsorbable in the body. The interbody device 400 of the described embodiments can be formed of a porous material or can be formed of a material that intrinsically participates in the growth of bone from one of adjacent vertebral bodies to the other of adjacent vertebral bodies. The interbody device 400 can be treated with, coated with, or used in combination with substances to inhibit scar tissue formation. The interbody device 400 of the described embodiments can be modified, or used in combination with materials to provide antibacterial properties, such as, but not limited to, electroplating or plasma spraying with silver ions or other substance. The antibacterial properties can include bactericidal and/or bacteriostatic characteristics. Similarly, anti-fungal characteristics can also be provided.

4. Interbody Guide Post

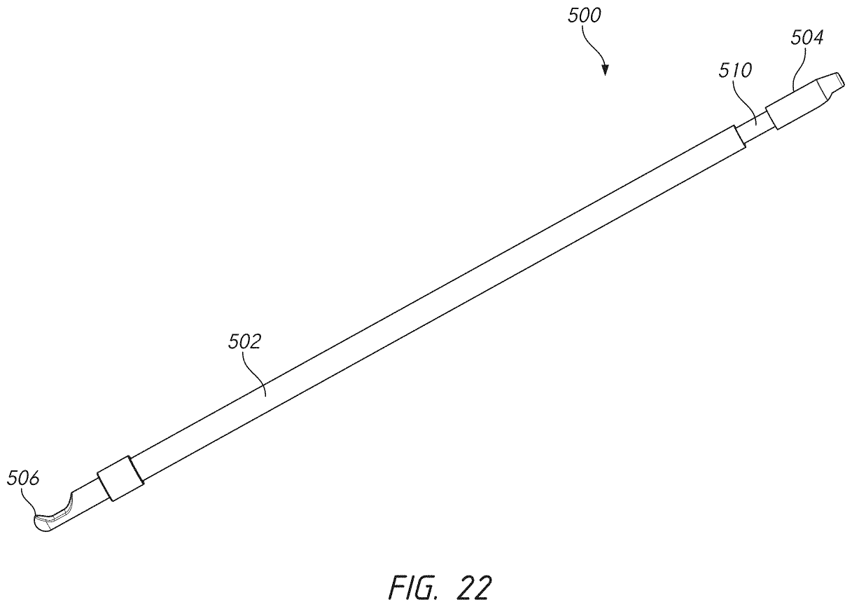

FIG. 22 is a side view of an embodiment of an interbody guide post 500. The interbody guide post 500 can include an interbody guide post shaft 502. The interbody guide post shaft 502 can include a proximal end 504 and a distal end 506. The distal end 506 can engage the opening 420 in the interbody device 400. In some embodiments, the distal end 506 is threaded. The distal end 506 can be rotated into engagement with the opening 420. The interbody guide post 500 can be limited in translational movement when coupled to the interbody device 400. In some embodiments, the distal end 506 is not threaded. The distal end 506 can be cylindrical in shape. The distal end 506 can function to center the interbody guide post 500 relative to the interbody device 400. In some embodiments, the interbody guide post 500 can freely rotate relative to the interbody device 400. In some embodiments, the interbody guide post 500 can freely translate relative to the interbody device 400. In some embodiments, the interbody guide post 500 can include an alignment feature 510. The alignment feature 510 can be a groove. The alignment feature 510 can be a circumferential groove. The alignment feature 510 can be a notch.

5. Drill

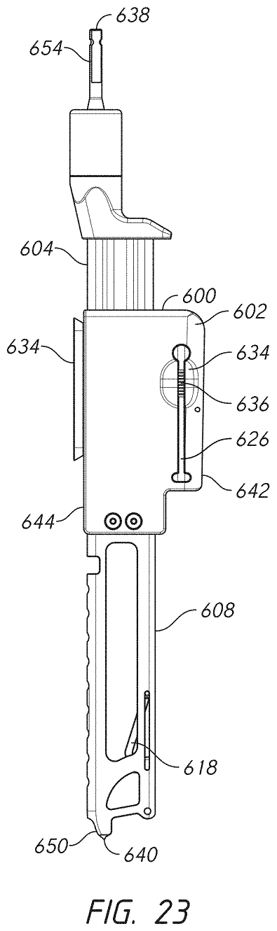

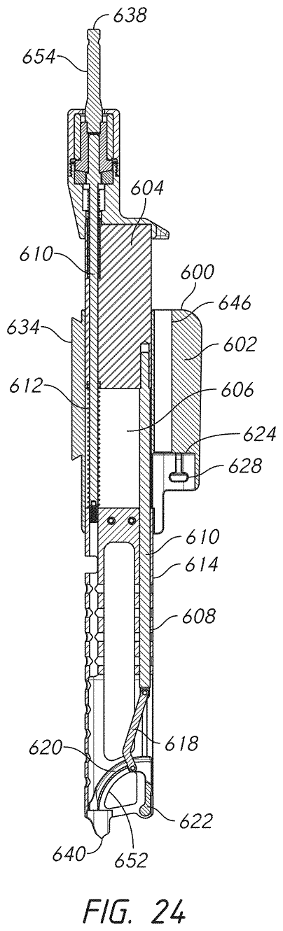

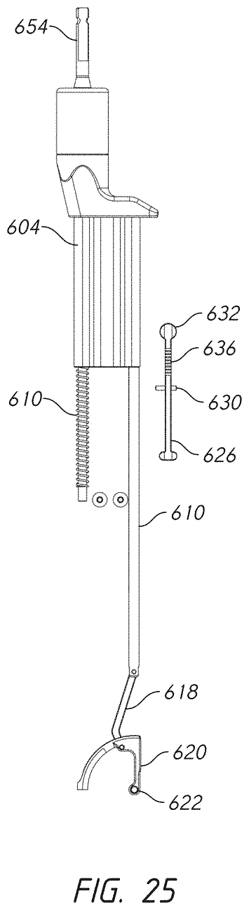

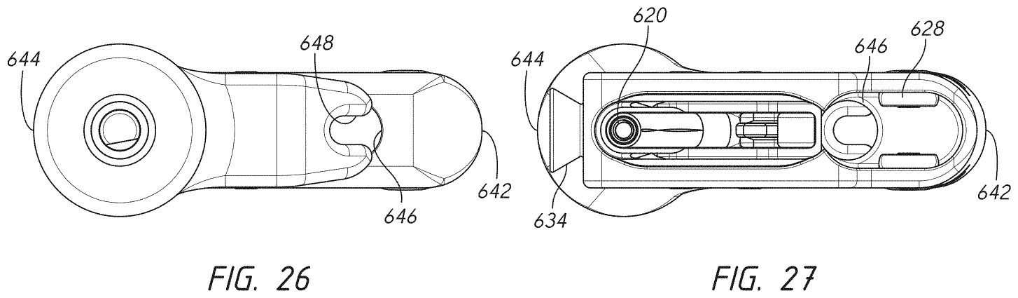

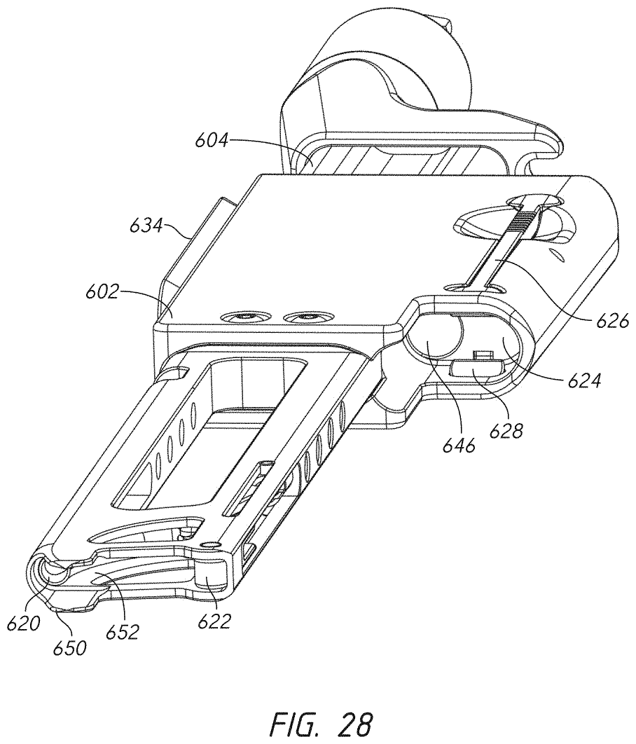

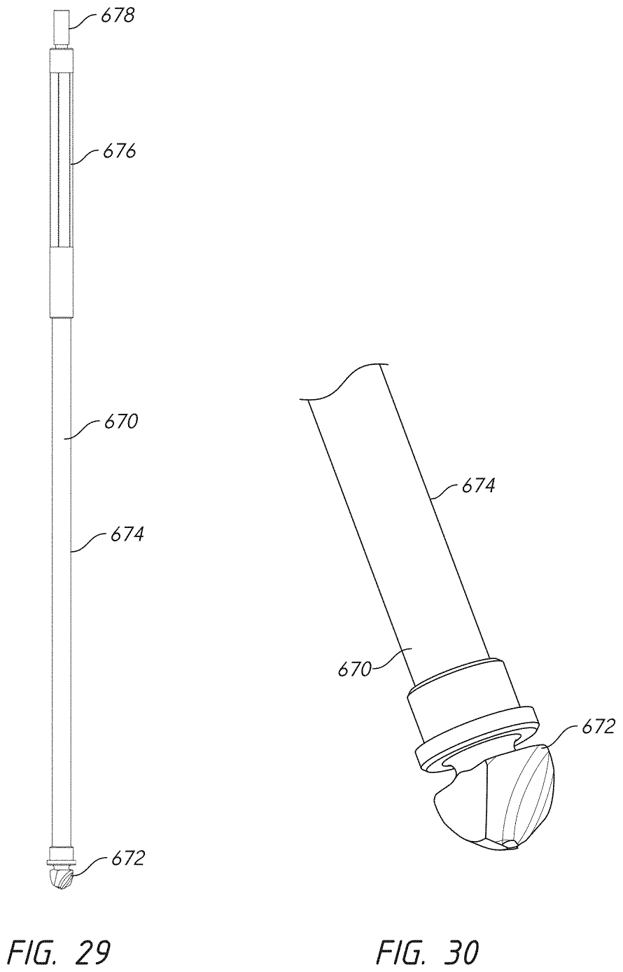

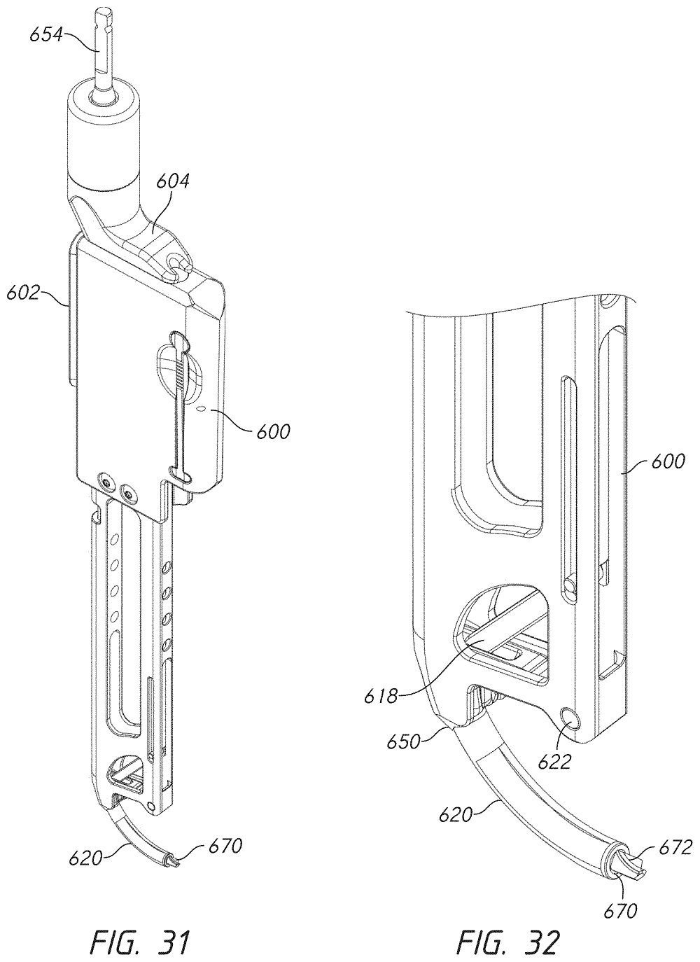

FIGS. 23-32 depict views of components for lumen preparation. FIG. 23 illustrates a front view of a drill 600. FIG. 24 illustrates a cross-sectional view of the drill 600. FIG. 25 illustrates a view of internal components of the drill 600. FIG. 26 illustrates a proximal view of the drill 600. FIG. 27 illustrates a distal view of the drill 600. FIG. 28 illustrates a perspective view of the drill 600. FIG. 29 illustrates a front view of a drill bit 670. FIG. 30 is a distal view of the drill bit 670. FIG. 31 is a perspective view of the drill 600 and the drill bit 670. FIG. 32 is a distal view of the drill 600 and the drill bit 670.

The drill 600 can include a drill handle 602. The drill handle 602 can include surfaces allowing a user to grip the drill 600. The drill 600 can include an advancer body 604. The drill handle 602 can be coupled to the advancer body 604. The drill handle 602 and the advancer body 604 can allow sliding between the drill handle 602 and the advancer body 604. The drill handle 602 and the advancer body 604 can comprise the same material. The drill handle 602 and the advancer body 604 can comprise different materials. In some embodiments, the advancer body 604 comprises a more rigid material such as one or more metals and the drill handle 602 comprises a more flexible material such as one or more polymers.

The drill handle 602 can include a cavity 606. The cavity 606 can extend from the proximal end of the drill handle 602. The cavity 606 can extend along the entire length of the drill handle 602, or a portion thereof. The advancer body 604 can be disposed within the cavity 606 of the drill handle 602. The advancer body 604 can extend proximally from the drill handle 602.

The drill 600 can include the drill body 608. The drill body 608 can extend distally from the drill handle 602. The drill handle 602 can be coupled to the drill body 608. The drill handle 602 and the drill body 608 can be coupled with one or more fasteners. The drill handle 602 can be integrally formed with the drill body 608. The drill handle 602 and the drill body 608 can comprise the same material. The drill handle 602 and the drill body 608 can comprise different materials. In some embodiments, the drill body 608 comprises a more rigid material such as one or more metals and the drill handle 602 comprises a more flexible material such as one or more polymers.

The drill 600 can include one or more pins 610. The drill 600 can include two pins 610. The two pins 610 can have different lengths. The two pins 610 can have different functions. The drill 600 can include one or more springs 612. The drill 600 can include one spring 612. The spring 612 can bias one pin 610. The one or more pins 610 and the one or more springs 612 can function as a suspension system. The drill 600 can include one or more channels 614. Each pin 610 can be disposed within the channel 614.

The advancer body 604 can slide downward within the cavity 606. As the advancer body 604 slides downward, the one or more pins 610 slide downward within the one or more channels 614. The one or more pins 610 are pushed by the advancer body 604. The one or more pins 610 can be separately formed from the advancer body 604. The one or more pins 610 and the advancer body 604 can be integrally formed. As the advancer body 604 slides downward, the one or more springs 612 can be compressed. The one or more springs 612 can bias the advancer body 604 upward. FIG. 36 illustrates the drill 600 with the drill body 608 removed.

The advancer body 604 is configured to advance a drill bit 670. The drill bit 670 can be loaded into the drill body 608. One of the pins 610 can engage the drill bit 670. The drill bit 670 can include a drill bit tip 672. The drill bit tip 672 can have a sharpened point. The drill bit tip 672 can include one or more flutes. The drill bit tip 672 can include a spiral blade. The drill bit 670 can include a drill bit shaft 674. The drill bit shaft 674 can be flexible to assume a curved shape. The drill bit 670 can include a keyed shaft 676. The drill bit 670 can include a proximal coupling 678. The proximal coupling 678 can engage one of the pins 610. In some embodiments, the pin 610 includes a threaded bore and the proximal coupling 678 includes a threaded post. In some embodiments, the pin 610 includes a keyed bore and can be configured to engage the keyed shaft 676. In some embodiments, the distal movement of the pin 610 can cause distal movement of the drill bit 670 as described herein. In some embodiments, the proximal movement of the pin 610 can cause proximal movement of the drill bit 670 as described herein. In some embodiments, the rotational movement of the pin 610 can cause rotational movement of the drill bit 670 as described herein. The drill 600 can be reusable. The drill tip 670 can be disposable.

The other pin 610 can be coupled to a linkage 618. The linkage 618 can be coupled to a swing arm 620. The swing arm 620 can be coupled to the drill body 608. The swing arm 620 can be coupled to a pivot bushing 622. The pivot bushing 622 can allow the swing arm 620 to rotate relative to the drill body 608. Downward motion of the pin 610 can cause the swing arm 620 to rotate about the pivot bushing 622. The swing arm 620 forms a portion of an arc. The swing arm 620 can guide the movement of the drill bit 670. The swing arm 620 can rotate relative to the pivot bushing 622 along a portion of an arc of 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, 180 degrees, 190 degrees, 200 degrees, 210 degrees, 220 degrees, 230 degrees, 240 degrees, 250 degrees, 260 degrees, 270 degrees, 280 degrees, 290 degrees, 300 degrees, 310 degrees, 320 degrees, 330 degrees, 340 degrees, 350 degrees, 360 degrees, or any range of two of the foregoing values.

The advancer body 604 can be released. The one or more springs 612 can bias the advancer body 604 upward. The one or more pins 610 can move upward. The movement of the pin 610 can cause corresponding movement of the linkage 618 and the swing arm 620. The movement of the pin 610 can cause the swing arm 620 to rotate in the opposite direction. The swing arm 620 can be retracted. The movement of the pin 610 can cause corresponding movement of the drill bit 670. The drill bit 670 can be retracted.

The drill 600 can include a latch cavity 624. The latch cavity 624. The latch cavity 624 can extend from the distal end of the tissue drill handle 602. The latch cavity 624 can have clearance for another component of the system.

The drill 600 can include one or more latch arms 626. The drill handle 602 can include the one or more latch arms 626. The drill 600 can include two latch arms 626. The latch arms 626 can be diametrically opposed. Each latch arm 626 can include a corresponding alignment feature 628. The corresponding alignment feature 628 can be a projection. The portal handle 302 can include one or more alignment features 314. The alignment features 314 can be diametrically opposed. Each alignment feature 314 of the portal 300 can be engaged by the corresponding alignment feature 628 of the drill 600.

The one or more latch arms 626 can be configured to pivot. The drill handle 602 can include pivot pins 630. Each latch arm 626 can be mounted on the pivot pin 630. The latch arm 626 can pivot relative to the pivot pin 630 to engage or disengage the corresponding alignment feature 628 from the alignment feature 510. The drill handle 602 can include a spring 632. The spring 632 can bias the corresponding alignment feature 628 of the latch arm 626 into engagement with the alignment feature 510 of the interbody guide post 500. The spring 632 can bias two latch arms 626. The spring 632 can bias the corresponding alignment features 628 of the latch arms 626 into engagement with the alignment features 510 of the interbody guide post 500. In some embodiments, the latch arms 626 do not engage the alignment feature 510 of the interbody guide post 500. The latch arms 626 can engage another component of the system.

The drill handle 602 can include one or more latch arm grooves 634. The drill handle 602 can include two latch arm grooves 634 corresponding to the two latch arms 626. The latch arm groove 634 can allow the user to pivot the corresponding latch arm 626. The latch arms 626 can include finger grips 636. The finger grips 636 can be depressed by the user to pivot the latch arms 626. The finger grips 636 can be depressed by the user to compress the spring 632. The latch arm 626 can pivot relative to the pivot pin 630. The latch arm 626 can pivot outward relative to the drill handle 602. The corresponding alignment feature 628 can disengage the alignment features 314 when the latch arms 626 are pivoted. The spring 632 can bias the latch arms 626 into engagement with the interbody guide post 500 when the latch arms 626 are released. The corresponding alignment features 628 of the latch arms 626 can lock with the alignment features 510 of the interbody guide post 500.

The drill 600 can include one or more corresponding sliding features 634. The sliding features 634 can engage another component of the system.

The drill 600 can include a proximal end 638 and a distal end 640. The drill 600 comprises a length between the proximal end 638 and the distal end 640. The length can be along the direction of insertion of the drill 600. The proximal end 638 can include the advancer body 604. The distal end 640 can include the drill body 608.

The drill 600 can include a first side 642 and a second side 644. The drill 600 can include a width extending between the first side 642 and the second side 644. The drill 600 can include a maximum width of 20 mm, 25 mm, 30 mm, 35 mm, 40 mm, 45 mm, 50 mm, or any range of two of the foregoing values. The drill 600 can include a thickness. The thickness can correspond to the transverse dimension of the width. The thickness can correspond to the transverse dimension of the first side 642. The thickness can correspond to the transverse dimension of the second side 644. The drill 600 can include a maximum thickness of 2 mm, 3 mm, 4 mm, 5 mm, 6 mm, 7 mm, 8 mm, 9 mm, 10 mm, 11 mm, 12 mm, 13 mm, 14 mm, 15 mm, or any range of two of the foregoing values. The width can be greater than the thickness of the drill 600.

The drill bit 670 can be inserted into the drill 600. The drill bit 670 can be inserted into the distal end 640 of the drill 600. The drill bit 670 can be inserted into the swing arm 620. The drill bit 670 can be inserted into the swing arm 620 when the swing arm is retracted. The drill bit 670 can be advanced into the drill body 608. The drill bit 670 can be advanced towards one of the pins 610. The drill bit 670 can engage the pin 610. The drill bit 670 and the pin 610 can be rotationally coupled. The drill bit 670 and the pin can be translationally coupled.

The drill 600 with the engaged drill bit 670 can be inserted over the interbody guide post shaft 502. The drill 600 can include a first lumen 646. The first lumen 646 can extend through the drill handle 602. The first lumen 646 can extend through the drill body 608. The advancer body 604 can form the first passageway 648. The first passageway 648 can form a circular arc. The circular arc can correspond to the diameter of the interbody guide post shaft 502. The drill 600 can slide relative to the interbody guide post shaft 502. The interbody guide post shaft 502 can be disposed within the first passageway 648 and the first lumen 646. The first lumen 646 can be positioned closer to the first side 642. The first passageway 648 can be positioned closer to the first side 642.

The interbody guide post shaft 502 can be in a fixed spatial relationship when engaged by the drill 600. The interbody guide post shaft 502 can be prevented from translation and rotation relative to the drill 600. When the interbody guide post shaft 502 and the drill 600 are coupled, the interbody guide post shaft 502 is a fixed distance relative to the pivot bushing 622 of the drill 600. The interbody guide post shaft 502 is in a pre-determined position relative to the swing arm 620 which pivots relative to the pivot bushing 622. The interbody guide post shaft 502 is in a pre-determined position relative to the arc formed by the swing arm 620. The interbody guide post shaft 502 is in a fixed position relative to the drill body 608 when the awl 530 and the drill 600 are coupled.

The drill body 608 can be shaped to engage the anatomy of the patient. The drill body 608 can include a ledge 650. The ledge 650 can include a tapered end. The ledge 650 can be shaped to rest against the vertebral body. The ledge 650 can be shaped to rest against the lateral side of the vertebral body. The ledge 650 can be shaped to engage the anatomy of the patient. The ledge 650 can be shaped to engage a generally curved surface. The ledge 650 can include a spike. The ledge 650 can be anchored to bone. The ledge 650 can stabilize the drill 600 against the anatomy of the patient. The drill body 608 can include a cavity 652. The cavity 652 can allow the swing arm 620 to rotate. The swing arm 622 can form an arc. The drill body 608 can include a shaped end for engaging the anatomy of the patient. The drill body 608 can include a shaped end for positioning relative to the anatomy.

The drill 600 can slide relative to the interbody guide post shaft 502. The drill 600 can slide from the proximal end 504 of the interbody guide post shaft 502. The drill 600 can lock into place relative to the interbody guide post shaft 502. The corresponding alignment feature 628 of the latch arm 626 can pivot into engagement with the alignment feature 510 of the interbody guide post shaft 502. The interbody guide post shaft 502 can extend past the first passageway 648 and the first lumen 646 of the drill 600.

The drill 600 and the drill bit 670 can form a lumen within bone. The swing arm 620 can rotate as the advancer body 604 is advanced. The swing arm 620 rotates relative to the pivot bushing 622. The drill bit shaft 674 can be flexible. The drill bit shaft 674 can follow the curved path of the swing arm 620 as the swing arm 620 advances in an arc. The drill bit 670 can be coupled to the pin 610. The pin 610 can be coupled to a drill coupler 654. The drill coupler 654 can rotate to rotate the drill bit 670. The drill coupler 654 can rotate the drill bit 670 indirectly through the pin 610. The drill coupler 654 can rotate the drill bit 670 at several thousand revolutions per minute. The drill coupler 654 can rotate the drill bit 670 to drill a lumen in bone.

The user sequentially advances and release the advancer body 604. The advancer body 604 advances the swing arm 620 in an arc. The drill bit 670 forms the hole. The advancer body 604 can be sequentially advanced and released relative to the drill handle 602 to drill the hole along the curved arc of the swing arm 620. The drill bit 670 can drill a lumen as the swing arm 620 rotates. The drill bit 670 can form the lumen. The drill bit shaft 674 can be flexible. The drill bit tip 672 can rotate to form a lumen along the arc of the swing arm 620. The swing arm 620 and the drill bit 670 can form a curved lumen within bone. The swing arm 620 can swing in an arc relative to the drill body 608. The swing arm 620 can carry the drill bit tip 672 and a portion of the drill tip shaft 674. The drill bit 670 can rotate relative to the swing arm 620 to bore a lumen. The drill bit 670 can be rotated by a drill motor coupled to the drill coupler 654. The drill coupler 654 can be disposed toward the proximal end of the drill 600.

The swing arm 620 can be moved in a circular arc by manipulation of the advancer body 604. FIGS. 31 and 32 show the fully advanced swing arm 620 and drill bit 670. The drill bit tip 672 can extend beyond the swing arm 620. The drill bit tip 672 can advance with the swing arm 620. The ledge 650 can be against bone, such that the lumen is formed within the bone. The swing arm 620 rotates relative to the pivot bushing 622. The swing arm 620 rotates upon the downward movement of the advancer body 604. The advancer body can abut the drill handle 602 when the swing arm 620 is fully advanced. The drill 600 can provide tactile feedback when the swing arm 620 is fully advanced. The swing arm 620 can form an arc. The swing arm 620 can form a complete circle. The swing arm 620 can extend through one vertebra. The swing arm 620 can extend through two vertebrae. The swing arm 620 can be moved between an advanced configuration and a retracted configuration. The swing arm 620 can be moved by the advancer body 604. The advancer body 604 moves the one or more pins 610 axially along the drill body 608. The downward manipulation of the advancer body 604 causes a longitudinal movement of the pin 610. In some embodiments, the advancer body 604 can be connected to the pin 610 directly, in which case the pin 610 is also manipulated by upward and downward motion of the advancer body 604. In other embodiments, the advancer body 604 can be connected to the pin 610 through mechanisms such as gears or hinges, wherein manipulation of the advancer body 604 translates into longitudinal movement of the pin 610. The pin 610 can be straight or curved or a combination of these shapes. The pin 610 causes movement of the linkage 618. The linkage 618 can be straight or curved or a combination of these shapes. The linkage causes movement of the swing arm 620. The other pin 610 causes movement of the drill bit 670. The drill bit 670 and the swing arm 620 simultaneously advance. The drill bit 670 and the swing arm 620 both advance with the movement of the advancer body 604.

Different curved shapes of the swing arm 620 are possible. In other embodiments, the swing arm 620 can have at least one straight segment and at least one curved segment. In some embodiments, the swing arm 620 is shaped to have a curved distal portion that has a desired arc so that the swing arm 620 follows a specified path when extended. In still other embodiments, a power source may be provided for hydraulic, pneumatic, or other power-assisted manipulation of the swing arm 620. The swing arm 620 can be generally curved to form a curved lumen. The swing arm 620 can be generally straight to form a straight lumen.

The swing arm 620 can comprise a tubular member. The swing arm 620 can include the rotating drill bit 670 disposed coaxially within. The rotating drill bit shaft 674 can be flexible. The rotating drill bit tip 672 can be guided by the swing arm 620. The swing arm 620 can have sufficient rigidity to guide the flexible rotating drill bit 670 into the shape of the desired lumen. The drill bit 670 can bend in the lateral direction. The drill bit 670 can create a curved cutting path. The drill bit 670 can be rotated by a power drill via drill coupler 654 to achieve the desired revolutions per minute to cut bone. The drill bit 670 can be advanced and retracted by the advancer body 604. The user can move the advancer body 604 distally until the advancer body 604 abuts the drill handle 602. The advancer body 604 can abut the drill handle 602 when the swing arm 620 is fully advanced. The advancer body 604 can abut the drill handle 602 when the swing arm 620 completes an arc. The advancer body 604 can abut the drill handle 602 when the swing arm 620 is aligned with the first lumen 646 of the drill 600. The advancer body 604 can abut the drill handle 602 when the swing arm 620 extends to the awl 530.

The swing arm 620 can be sized to be able to pass through the vertebral body. The swing arm 620 can be sized to be able to pass through two vertebral bodies. The resulting hole from the swing arm 610 and the drill bit 670 is sized for the bone tie 100, 200, 300 to be inserted. The swing arm 620 can have a diameter in the range of about 1 mm to 5 mm, preferably about 2 mm to 4 mm, and most preferably about 3 mm. The end of the drill bit 670 can include a cutting surface for creating the lumen. The drill bit 670 can be of any appropriate configuration and with any number of points. In some embodiments, the drill bit 670 may be round, flat, beveled or stepped.

The drill bit 670 can be connected to the drill coupler 654 to provide the axial rotation. The drill coupler 654 can have a configuration that is complementary to a coupling of a powered drill. In some embodiments, the drill coupler 654 can have a feature to provide an anti-rotational connection, such as for example a flat surface, or a shaft having a square or hexagonal cross-section. The drill coupler 654 can rotate one pin 610. The pin 610 can function as a drive shaft. The drill coupler 654 can rotate the drill bit 670.

The drill 600 can be used by positioning the drill bit 670 against the vertebral body. When the drill 600 is actuated and the drill bit 670 is rotated, the swing arm 620 forms a curved arc and the drill bit 670 cuts through both the vertebral bodies. The drill bit 670 forms a predicable path through bone.

Once the lumen is formed, the swing arm 620 and the drill bit 670 can be retracted by releasing the advancer body 604. The swing arm 620 can retract under the influence of the spring 612. The lumen can be utilized with the bone tie 100, 200, 300 to anchor or stabilize the facet joint. The lumen can be utilized with the bone tie 100, 200, 300 to alter the spacing or motion between adjacent vertebrae. The drill 600 can be removed, leaving the interbody guide post shaft 502 and the interbody device 400.

6. Methods of Use

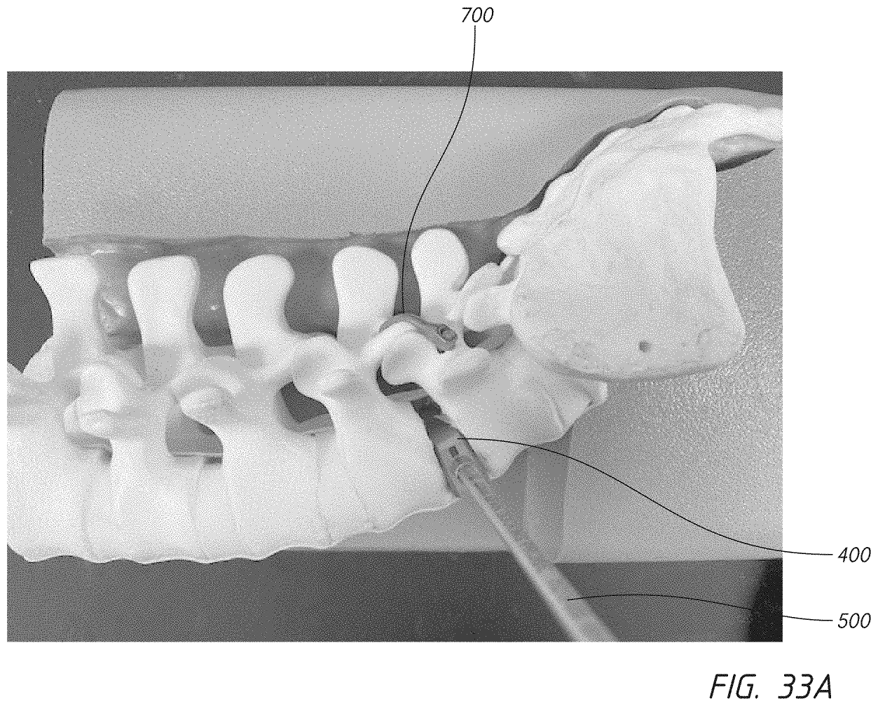

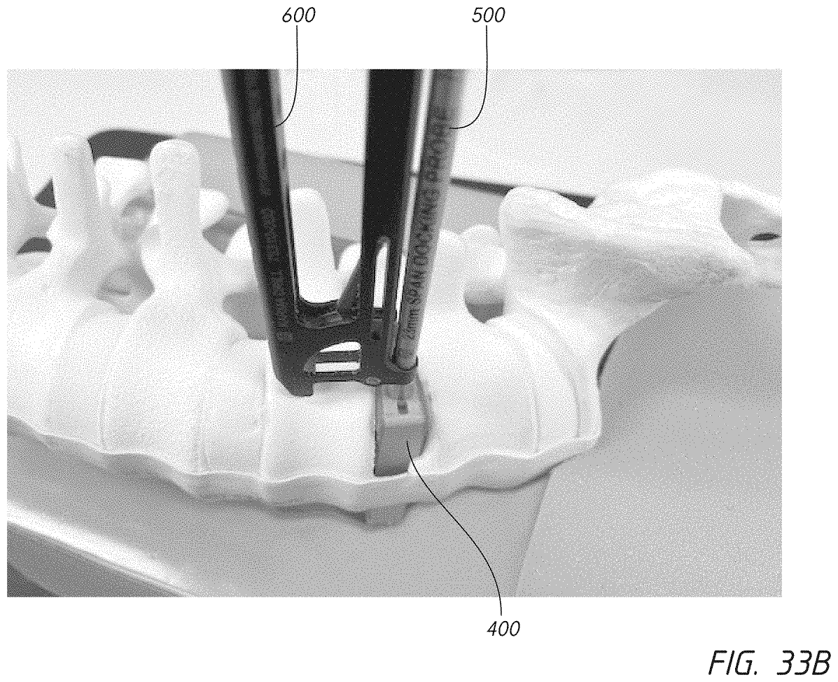

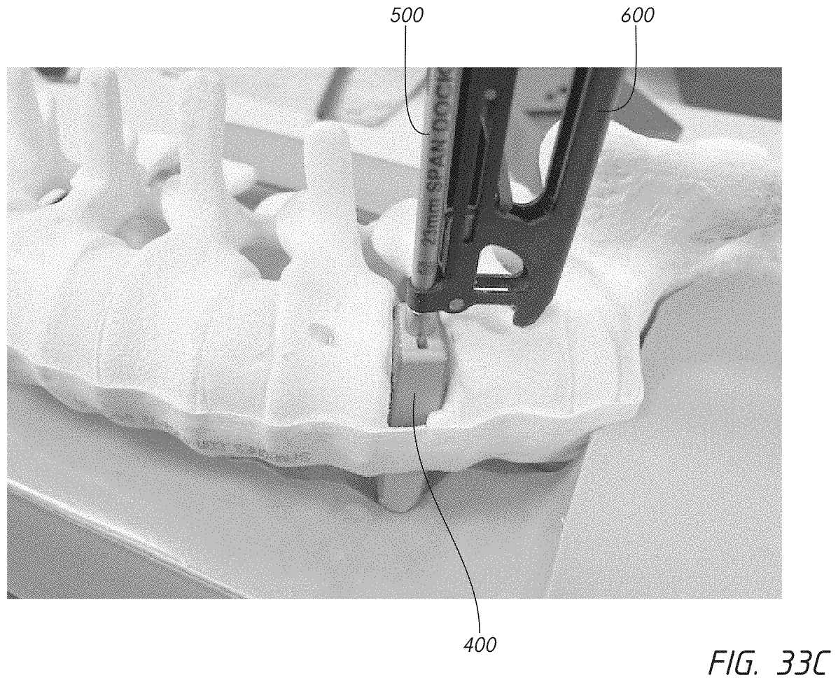

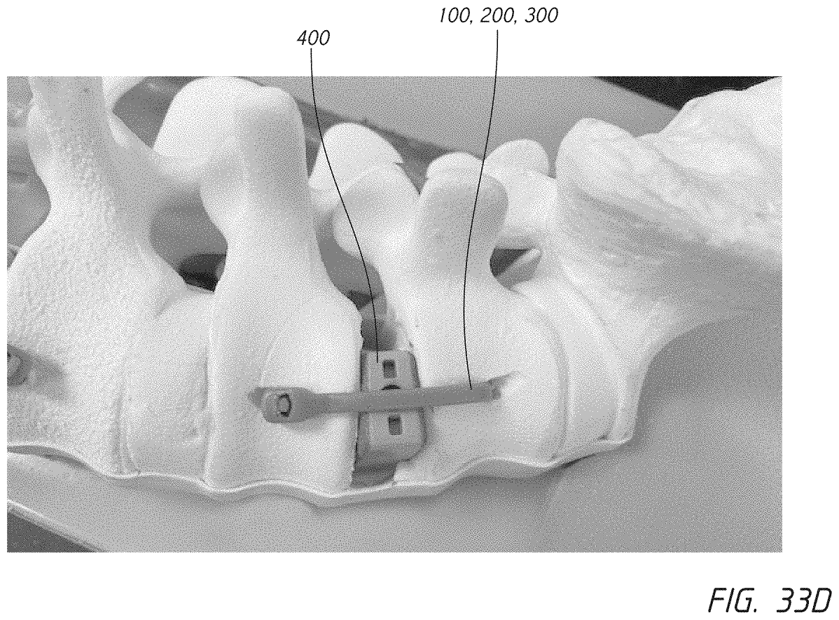

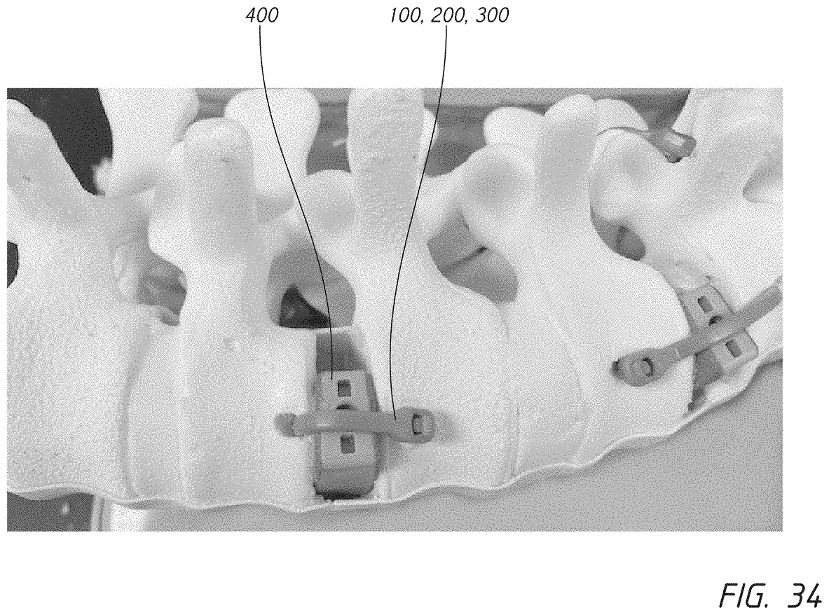

FIG. 18 is a side view of an embodiment of the interbody device 400 and the bone tie 100, 200, 300. FIG. 33A-33D are views of methods. FIG. 34 is a view of a method. The methods of use can include any steps described herein. The methods of use can include steps in any order. The interbody device 400 can be utilized with one bone tie 100, 200, 300. The interbody device 400 can be utilized with two bone ties 100, 200, 300. The interbody device 400 can be utilized with a plurality of bone ties 100, 200, 300. In some embodiments, the bone tie 100, 200, 300 goes through a portion of the vertebra. In some embodiments, the bone tie 100, 200, 300 goes through a portion of the interbody device 400. In some embodiments, the bone tie 100, 200, 300 and the interbody device 400 facilitate spinal fusion.

The intervertebral space can be debrided. In some methods of use, a portion of the surface of the vertebrae can be prepared for fusion. In some methods of use, a portion of the surface of the vertebrae can be ground, scored, roughened, or sanded, such that the surface of the vertebra can better adhere to any substances to aid in fusion and/or otherwise fuse more readily to the interbody device 400. In some methods of use, the surgical procedure can include preparing the area near and/or around the vertebra by, for example, removing all or a portion of ligaments, cartilage, and/or other tissue. In some methods of use, the area can be prepared by removing all or a portion of the disc.

In some embodiments, the interbody device 400 can be packed with natural or artificial bone matrix and/or other osteogenesis factors and inserted into the intervertebral space. The interbody device 400 can include one or more windows 416. The windows 416 can be packed with material. The window 416 can be packed with material before insertion into the intervertebral space. The window 416 can be packed with material after insertion into the intervertebral space. The window 416 can be packed with material after insertion into the intervertebral space through the opening 420. The window 416 can be packed with material after insertion into the intervertebral space through the one or more features 422. Two or more windows 416 can be packed before insertion into the intervertebral space. One of the two or more windows 416 can be packed before insertion into the intervertebral space and one of the two or more windows 416 can be packed after insertion into the intervertebral space. In some methods, the interbody device 400 can be packed with material before insertion into the intervertebral space. In some methods, the interbody device 400 can be packed with material after insertion into the intervertebral space.

The interbody device 400 can be coupled to the interbody guide post 500. The distal end 506 of the interbody guide post shaft 502 can engage the opening 420 in the interbody device 400. In some embodiments, the distal end 506 of the interbody guide post 500 can engage one or more features 422. In some embodiments, the interbody guide post 500 can engage one or more undercuts 424. The interbody device 400 can be rotationally coupled to the interbody guide post 500. The interbody device 400 can be translationally coupled to the interbody guide post 500. The interbody guide post 500 can include an impaction surface. The interbody guide post 500 can be struck to advance the interbody device 400 into the intervertebral space.

The interbody device 400 can be inserted into the intervertebral space. The interbody device 400 can be inserted between vertebrae. In some methods, the interbody device 400 can be inserted between a superior vertebra and an inferior vertebra. The interbody device 400 can include any features described herein. The interbody device 400 can be inserted via a posterior approach. The interbody device 400 can be inserted via a lateral approach. The interbody device 400 can be inserted via an anterior approach. The interbody device 400 can be inserted via any approach. The interbody device 400 can be inserted with any insertion tool. The interbody device 400 can be positioned between vertebrae. FIGS. 12, 33A-33D, and 34 illustrates an embodiment of the interbody device 400 positioned between vertebrae.

The drill 600 can be coupled to the interbody guide post 500. The drill 600 can slide down the interbody guide post shaft 502 toward the interbody device 400. The distal end 506 of the interbody guide post shaft 502 can be engaged with the opening 420. The interbody device 400 can be rotationally coupled to the interbody guide post 500 as the drill 600 is advanced. The interbody device 400 can be translationally coupled to the interbody guide post 500 as the drill 600 is advanced. The interbody device 400 can be inserted into the intervertebral space as the drill 600 is advanced along the interbody guide post shaft 502. The drill 600 can be in a fixed relationship to the interbody guide post 500. The drill 600 can be in a fixed relationship to the interbody device 400.

The method can include drilling a curved lumen. The drill 600 can be slid along the interbody guide post shaft 502 until it locks in place. The latch arms 626 of the drill 600 can engage the alignment feature 510 of the interbody guide post shaft 502. The method can include attaching a power drill to the drill coupler 654. The drill 600 can include the drill bit 670. The drill bit 670 can be guided by the swing arm 620. The advancer body 604 can be moved proximally and distally to advance and retract the swing arm 620. The swing arm 620 and the drill bit 670 can form a curved lumen. The drill 600 can form a curved lumen from the outer surface of the vertebral body to the intervertebral space. The drill 600 can form a curved lumen from the outer surface of the vertebral body and at least partially into the interbody device 400. The drill 600 can form a curved lumen from the outer surface of the vertebral body and through the interbody device 400. The drill 600 can form a curved lumen from the outer surface of the vertebral body and through window 416. The drill 600 can form a curved lumen from the outer surface of the vertebral body and through the notch 418. The drill 600 can form a curved lumen from the outer surface of the vertebral body and through graft material located within the window 416. The drill 600 can form a curved lumen from the outer surface of the vertebral body and through graft material located within the notch 418. The drill 600 can form a curved lumen from the outer surface of the vertebral body and at least to a midpoint of the interbody device 400. In some methods, the drill 600 can form a curved hole from the outer surface of the vertebral body to the outer surface of the adjacent vertebra. In some methods of use, a lumen is formed through the vertebra. The lumen can be formed with the drill 600, or a lumen-forming tool, such as a tissue punch or reamer. The lumen is formed through the vertebral body. In some embodiments, the lumen is formed through cortical bone. The lumen is formed through the dense outer shell of the vertebral body. In some embodiments, the lumen is not formed through the cancellous bone. In some embodiments, the lumen is formed through cancellous bone. In some methods of use, the lumen is formed through the vertebra before placement of the interbody device 400. In some methods of use, the lumen is formed through the vertebra after placement of the interbody device 400. In some methods of use, the lumen is formed through the notch 418. In some methods of use, the lumen is formed through the window 416. In some methods of use, the lumen is formed through graft material. In some methods of use, the lumen is formed through material packed in the interbody device 400. In some methods of use, the lumen is formed through the vertebra to align with the notch 418 of the interbody device 400. In some methods of use, the lumen is formed through the vertebra to align with the window 414 of the interbody device 400.

In some methods of use, a lumen is formed to facilitate implantation of the bone tie 100, 200, 300. In some embodiments, at least a portion of the lumen has a curved or non-linear configuration. In some embodiments, at least a portion of the lumen has a straight or linear configuration. In some methods of use, two or more lumens are formed. In some methods of use, a lumen is formed in the superior vertebra and a lumen is formed in the inferior vertebra. In some methods of use, two lumens are formed in the superior vertebra. In some methods of use, two lumens are formed in the inferior vertebra. A drill or other device can be used to form the lumen. In some embodiments, one lumen-forming tool forms one or more lumens. In some embodiments, two lumen-forming tools are utilized to form two lumens.

FIG. 18 illustrates a method of forming two lumens. The interbody device 400 can be inserted into the intervertebral space before the lumens are formed. The drill 600 can be positioned relative to the first vertebral body. The drill bit 670 can form a first lumen through the first vertebral body to the first notch 418. The drill bit 670 can form a first lumen from the vertebral body to the superior surface 406. The drill bit 670 can extend through the first notch 418. The drill bit 670 can extend to the proximal surface 414. The drill bit 670 can extend through the channel formed by the first notch 418. The drill bit 670 can extend through a closed channel. The drill bit 670 can form a lumen through graft material that extends into the first notch 418. The drill bit 670 can extend through an open channel. The drill bit 670 can form a continuous arc from the first vertebral body through the first notch 418. The drill bit 670 can form a lumen from the outer surface of the first vertebral body to the superior surface 406. The drill bit 670 can be retracted.

The drill 600 can be positioned relative to the second vertebral body. The drill bit 670 can form a second lumen through the second vertebral body to the second notch 418. The drill bit 670 can form a second lumen from the vertebral body to the inferior surface 408. The drill bit 670 can extend through the second notch 418. The drill bit 670 can extend to the proximal surface 414. The drill bit 670 can extend through the channel formed by the second notch 418. The drill bit 670 can extend through a closed channel. The drill bit 670 can form a lumen through graft material that extends into the second notch 418. The drill bit 670 can extend through an open channel. The drill bit 670 can form a continuous arc from the second vertebral body through the second notch 418. The drill bit 670 can form a lumen from the outer surface of the second vertebral body to the inferior surface 408. The drill bit 670 can be retracted.

Other methods are contemplated. In some embodiments, the drill 600 can be positioned relative to the proximal end 414 of the interbody device 400. The drill bit 670 can extend from the proximal end 414 through the first notch 418. The notch 418 can guide the drill bit 670 during initial placement. The drill bit 670 can form a lumen from the superior surface 406 to the outer surface of the first vertebral body. The drill 600 can be repositioned relative to the proximal end 414 of the interbody device 400. The drill bit 670 can extend from the proximal end 414 through the second notch 418. The notch 418 can guide the drill bit 670 during initial placement. The drill bit 670 can form a lumen from the inferior surface 408 to the outer surface of the second vertebral body. In some embodiments, the notch 418 is curved to match the curvature of the drill 600.

In some embodiments, the drill 600 can be positioned relative to the first vertebral body and forms an arc. The drill 600 can be positioned relative to the second vertebral body and forms an arc. In some embodiments, the interbody device 400 can be inserted into the intervertebral space after the lumens are formed.

In some embodiments, the drill 600 can be positioned relative to the intervertebral space and forms an arc through the first vertebral body. The drill 600 can be positioned relative to the intervertebral space and forms an arc through the second vertebral body. In some embodiments, the interbody device 400 can be inserted into the intervertebral space after the lumens are formed.

FIGS. 33A-33D illustrate methods of forming a lumen. The interbody device 400 can be inserted into the intervertebral space with the interbody guide post 500. The interbody guide post 500 is docked to the interbody device 400 in FIG. 33A. In some embodiments, the interbody device 400 can be inserted into the intervertebral space before the interbody guide post 500 is docked to the interbody device 400.

The drill 600 is docked to the interbody guide post 500 in FIG. 33B. The drill 600 slides relative to the interbody guide post shaft 502. The latch arm 626 of the drill 600 can engage the corresponding alignment feature 628 with the alignment feature 510 of the interbody guide post 500. The drill 600 can be perpendicular to the proximal end 404 of interbody device 400 when the latch arm 626 engages the interbody guide post 500. In some embodiments, the drill 600 can be in one of two fixed orientations relative to the interbody device 400 when the latch arm 626 engages the interbody guide post 500. The drill 600 can be positioned relative to the first vertebral body. The drill bit 670 can form a first lumen through the first vertebral body. The drill bit 670 can form a first lumen from the vertebral body to the superior surface 406 of the interbody device 400. The drill bit 670 can form a first lumen from the vertebral body to the notch 418. The drill bit 670 can form a first lumen from the vertebral body to the window 416. The drill bit 670 can extend to a surface of the interbody device 400. The drill bit 670 can form a continuous arc from the first vertebral body to a portion of the interbody device 400. In some embodiments, drill bit 670 can extend through the notch 418. In some embodiments, drill bit 670 can extend through the window 416. In some embodiments, drill bit 670 can extend through the intervertebral space. The drill bit 670 can extend through a closed channel. The drill bit 670 form a lumen through graft material. The drill bit 670 can extend through an open channel. The drill bit 670 can form a continuous arc from the first vertebral body through at least a portion of the interbody device 400. The drill bit 670 can be retracted.

The latch arm 626 of the drill 600 can disengage the corresponding alignment feature 628 with the alignment feature 510 of the interbody guide post 500. The drill 600 can be rotated relative to the interbody guide post shaft 502. The drill 600 can be rotated 180 degrees relative to the interbody guide post shaft 502. The drill 600 is docked to the interbody guide post 500 in FIG. 33C. The latch arm 626 of the drill 600 can engage the corresponding alignment feature 628 with the alignment feature 510 of the interbody guide post 500. The drill 600 can be perpendicular to the proximal end 404 of the interbody device 400 when the latch arm 626 engages the interbody guide post 500. In some embodiments, the drill 600 can be in the other of two fixed orientations relative to the interbody device 400 when the latch arm 626 engages the interbody guide post 500. The drill 600 can be positioned relative to the second vertebral body. The drill bit 670 can form a second lumen through the second vertebral body. The drill bit 670 can form a second lumen from the vertebral body to the inferior surface 408 of the interbody device 400. The drill bit 670 can form a second lumen from the vertebral body to the notch 418. The drill bit 670 can form a second lumen from the vertebral body to the window 416. The drill bit 670 can extend to a surface of the interbody device 400. The drill bit 670 can form a continuous arc from the second vertebral body to a portion of the interbody device 400. In some embodiments, drill bit 670 can extend through the notch 418. In some embodiments, drill bit 670 can extend through the window 416. In some embodiments, drill bit 670 can extend through the intervertebral space. The drill bit 670 can extend through a closed channel. The drill bit 670 form a lumen through graft material. The drill bit 670 can extend through an open channel. The drill bit 670 can form a continuous arc from the second vertebral body through at least a portion of the interbody device 400. The arcs formed from the drill 600 can intersect. The arcs formed from the drill 600 can form a crossing pattern. The arcs formed from the drill 600 can form a continuous through channel. The drill bit 670 can be retracted. The drill 600 can have a first orientation relative to the first vertebral body while forming the first lumen. The drill 600 can have a second orientation relative to the second vertebral body while forming the second lumen. The drill 600 can be coupled relative to the interbody device 400 while forming the lumens.

FIG. 33D illustrates the bone tie 100, 200, 300 passed through the lumens formed by the drill 600. The bone tie 100, 200, 300 can be passed through a curved lumen in the first vertebra. The bone tie 100, 200, 300 can be passed through the notch 418. The bone tie 100, 200, 300 can be passed through the window 416. The bone tie 100, 200, 300 can be passed through the notch 418 and the window 416. The bone tie 100, 200, 300 can be passed through a linear pathway through the interbody device 400. The bone tie 100, 200, 300 can be passed through a curved pathway through the interbody device 400. The bone tie 100, 200, 300 can be passed through a closed pathway through the interbody device 400. The bone tie 100, 200, 300 can be passed through a partially open pathway through the interbody device 400. The bone tie 100, 200, 300 can be passed through a curved lumen in the second vertebra. The method can include docking the interbody guide post 500. The method can include docking the drill 600 to the interbody guide post 500. The method can include drilling a first lumen. The method can include rotating the drill 600 relative to the interbody guide post 500. The method can include removing the drill 600. The method can include docking the drill 600 to the interbody guide post 500 in another orientation. The method can include drilling a second lumen. The method can include inserting the bone tie in the first lumen and the second lumen. The method can include securing the bone tie.

FIG. 34 illustrates the bone tie 100, 200, 300 passed through a lumen formed by the drill 600. The drill 600 can form a continuous arc from the first vertebra to the second vertebra. The drill 600 can form a continuous arc without being repositioned. The drill 600 is repositioned in FIGS. 33A-33D. In FIG. 34, the drill 600 is not repositioned.

The drill 600 can be positioned relative to the first vertebral body. The drill bit 670 can form a lumen through the first vertebral body. The drill bit 670 can extend through the interbody device 400. The drill bit 670 can extend through the notch 418. The drill bit 670 can extend through the window 416. The drill bit 670 can extend from the superior surface 406 to the inferior surface 408. The drill bit 670 can extend through the intervertebral space. The drill bit 670 can extend through a closed channel. The drill bit 670 can form a lumen through graft material. The drill bit 670 can extend through an open channel. The drill bit 670 can form a lumen through the second vertebral body. The drill bit 670 can form a continuous arc from the first vertebral body to the second vertebral body. The drill 600 can have a single orientation relative to the first vertebral body while forming the lumen. The drill 600 can have a single orientation relative to the second vertebral body while forming the lumen. The drill 600 can have a single orientation relative to the interbody device 400 while forming the lumen.

The method can include inserting the interbody device 400 into the intervertebral space. The interbody guide post 500 can be docked to a first vertebra. In some embodiments, a hole is formed in the first vertebra. In some embodiments, at least a portion of the hole has a straight or linear configuration. The hole can extend downward from a surface of the first vertebra. The interbody guide post 500 can be inserted into the hole. In some embodiments, the interbody device 400 can be inserted into the intervertebral space before the interbody guide post 500 is docked to the first vertebra. In some embodiments, the interbody device 400 can be inserted into the intervertebral space after the interbody guide post 500 is docked to the first vertebra. The interbody guide post 500 can be inserted directly into a vertebral body.

The method can include docking the drill 600 to the interbody guide post 500. The drill 600 can slide relative to the interbody guide post shaft 502. The latch arm 626 of the drill 600 can engage the corresponding alignment feature 628 with the alignment feature 510 of the interbody guide post 500. The drill 600 can extend toward the second vertebra when the latch arm 626 engages the interbody guide post 500. In some embodiments, the drill 600 can be in a fixed orientation relative to the interbody guide post 500 when the latch arm 626 engages the interbody guide post 500. The drill bit 670 can be positioned relative to the second vertebral body when the latch arm 626 engages the interbody guide post 500. The drill bit 670 can form a lumen through the second vertebral body. The drill bit 670 can form a lumen from the second vertebral body to the inferior surface 408 of the interbody device 400. The drill bit 670 can form a lumen from the second vertebral body to the notch 418. The drill bit 670 can form a lumen from the vertebral body to the window 416. The drill bit 670 can extend through the interbody device 400. In some embodiments, drill bit 670 can extend through the notch 418. In some embodiments, drill bit 670 can extend through the window 416. In some embodiments, drill bit 670 can extend through the intervertebral space. The drill bit 670 can extend through a closed channel. The drill bit 670 form a lumen through graft material. The drill bit 670 can extend through an open channel. The drill bit 670 can form a continuous arc through the interbody device 400. The drill bit 670 can form a continuous arc from the second vertebral body to the first vertebral body. The drill bit 670 can make a single pass through the second vertebral body to the first vertebral body. The drill bit 670 can form a continuous arc from the second vertebral body to the hole. The drill bit 670 can form a continuous arc toward the interbody guide post 500. In some embodiments, the drill bit 670 can form a lumen back to the interbody guide post 500. Other methods are contemplated. In some embodiments, the interbody guide post 500 can be docked to a second vertebra. In some embodiments, the drill bit 670 can make a single pass through the first vertebral body to the second vertebral body.

The bone tie 100, 200, 300 can be passed through a curved lumen in the first vertebra. The bone tie 100, 200, 300 can be passed through the notch 418. The bone tie 100, 200, 300 can be passed through the window 416. The bone tie 100, 200, 300 can be passed through the notch 418 and the window 416. The bone tie 100, 200, 300 can be passed through a linear pathway through the interbody device 400. The bone tie 100, 200, 300 can be passed through a curved pathway through the interbody device 400. The bone tie 100, 200, 300 can be passed through a closed pathway through the interbody device 400. The bone tie 100, 200, 300 can be passed through a partially open pathway through the interbody device 400. The bone tie 100, 200, 300 can be passed through a curved lumen in the second vertebra. The bone tie 100, 200, 300 can be passed through a continuous arc from the first vertebral body to the second vertebral body.

In some embodiments, one or more bone ties 100, 200, 300 may be used via one or more lumens. In some embodiments, one or more bone ties 100, 200, 300 may be used via one or more channels. In some embodiments, one or more bone ties 100, 200, 300 may be used to engage one or more notches 418. In some embodiments, one or more bone ties 100, 200, 300 may be used to engage one or more windows 416. In some embodiments, the lumens may be placed at angles to allow “X” crossing pattern of bone ties 100, 200, 300. In some embodiments, the notches 414 may be placed at angles to allow “X” crossing pattern of bone ties 100, 200, 300.

In some embodiments, the bone tie 100, 200, 300 intersects the interbody device 400. The bone tie 100, 200, 300 can be used in addition to a facet bone tie 700. The bone tie 100, 200, 300 can be used in place of facet bone tie 700 shown in FIG. 33A. The bone tie 100, 200, 300 can be used with an advancer system. The bone tie 100, 200, 300 can be used with a retriever system. The bone tie 100, 200, 300 can be used with a portal system.

The bone tie 100, 200, 300 can form a small loop as shown in FIG. 18. The bone tie 100, 200, 300 can form a larger loop as shown in FIG. 34. The bone tie 100, 200, 300 can form a still larger loop as shown in FIG. 33D. The bone tie 100, 200, 300 can extend along a continuous arc. The bone tie 100, 200, 300 can extend along a discontinuous arc.

The distal end of the bone tie 100, 200, 300 can be passed through a portion of the interbody device 400. The distal end of the bone tie 100, 200, 300 can be inserted into the window 416. FIG. 19 illustrates an embodiment of the interbody device 400 where the bone tie 100, 200, 300 can be inserted into the window 416. The bone tie 100, 200, 300 can extend through the window 416. The bone tie 100, 200, 300 can extend through the graft chamber. The bone tie 100, 200, 300 can extend through a lumen formed in the graft material. The interbody device 400 can omit the notch 418. The bone tie 100, 200, 300 can pass through any portion of the interbody device 400.

The distal end of the bone tie 100, 200, 300 can be inserted into the notch 418. FIGS. 12-18, 20, and 21 illustrates an embodiment of the interbody device 400 with the notch 418. The bone tie 100, 200, 300 can extend through the notch 418. The bone tie 100, 200, 300 can extend through the notch 416. The notch 416 can be sized and shaped to accept the bone tie. In some embodiments, the bone tie 100, 200, 300 does not pass through the window 416. The notch 416 can be partially enclosed. The notch 416 can form a closed channel. The notch 416 can form a continuous perimeter. In some embodiments, the bone tie 100, 200, 300 passes through the notch 418 and the window 416. The bone tie 100, 200, 300 can extend through a lumen formed in the graft material. The notch 416 can be partially open. The notch 416 can form an open channel. The notch 416 can form a discontinuous perimeter. In some embodiments, the interbody device 400 can omit the window 416. The notch 418 can provide a distinct or dedicated pathway for the bone tie 100, 200, 300. The notch 418 can extend through the interbody device 400, or a portion thereof. The notch 418 can be angled toward a vertebral body. The notch 418 can be angled toward two vertebral body. The notch 418 can be angled toward the arc to be formed by the drill 600. The notch 418 can be angled toward the lumen in the vertebra.

FIGS. 12 and 21 illustrates another embodiment of the interbody device 400 with notches 418. The first bone tie 100, 200, 300 can extent through a first notch 418 toward the superior vertebra. The second bone tie 100, 200, 300 can extent through a second notch 418 toward the inferior vertebra. The notches 418 can be slanted. The notches 418 can be curved. The notches 418 can be angled toward the lumens in the vertebrae.

The distal end of the bone tie 100, 200, 300 can be inserted into the lumen of the vertebra. In some methods, the bone tie 100, 200, 300 is threaded through a lumen of the superior vertebral body. In some methods, the bone tie 100, 200, 300 is threaded through a lumen of the inferior vertebral body. In some methods, the bone tie 100, 200, 300 is threaded through the lumen of the superior vertebra and a second bone tie 100, 200, 300 is threaded through a lumen of the inferior vertebra. In some methods, the bone tie 100, 200, 300 is passed through the interbody device 400 before passing through the lumen in the vertebra. In some methods, the bone tie 100, 200, 300 is passed through the lumen in the vertebra before passing through the interbody device 400.

The distal end of the bone tie 100, 200, 300 can be inserted into the fastener section 106, 206, 306. In some embodiments, the bone tie 100, 200, 300 comprises a strap with gears 128, 228, 328 along the length of the bone tie 100, 200, 300. The gears 128, 228, 328 interact with the fastener section 106, 206, 306. The fastener section 106, 206, 306 can include any latching mechanism. The fastener section 106, 206, 306 can include the ratchet 122, 222, 322. The distal end of the bone tie 100 can be cut before passage through the fastener section 106. The head 136 can be removed. The distal end of the bone tie 100, 200, 300 can be tapered and smooth to facilitate passage of the bone tie through or around anatomical structures to be secured. The distal end of the bone tie 100, 200, 300 is fed through the fastener section 106, 206, 306. The loop is made consecutively smaller by pulling the bone tie 100, 200, 300 through the fastener section 106, 206, 306. Once the desired tension is achieved, the excess portion of the bone tie 100, 200, 300 can be cut and discarded. The interbody device 400 is retained within the loop of the bone tie 100, 200, 300. A portion of the vertebra is retained within the loop of the bone tie 100, 200, 300.

The bone tie 100, 200, 300 can secure the interbody device 400 to the vertebra. In some embodiments, two or more bone ties 100, 200, 300 can secure the interbody device 400 to vertebrae. The bone tie 100, 200, 300 and the interbody device 400 can be configured to fuse together two or more vertebrae of the vertebral column. The bone tie 100, 200, 300 and the interbody device 400 can be configured for spinal fusion. In some embodiments, the bone tie 100, 200, 300 and the interbody device 400 can be utilized in any level in the spine. In some embodiments, the bone tie 100, 200, 300 and the interbody device 400 can maintain the anatomical spacing between vertebrae. The bone tie 100, 200, 300 can be a retaining member for anchoring the interbody device 400 within the intervertebral space. In some embodiments, the bone tie 100, 200, 300 can pass through the notch 418 or the window 416 of the interbody device 400 when the interbody device 400 is inserted within the intervertebral space. The bone tie 100, 200, 300 can be adapted for securing the location of the interbody device 400 with respect to at least one vertebra. The bone tie 100, 200, 300 can be adapted for securing the location of the interbody device 400 with respect to adjacent vertebrae.

The bone tie 100, 200, 300 and the interbody device 400 can be configured for altering the motion of the vertebral column. In some embodiments, the bone tie 100, 200, 300 and the interbody device 400 can limit or reduce motion of a vertebra. In some embodiments, the bone tie 100, 200, 300 and the interbody device 400 can limit or reduce motion of adjacent vertebrae. In some embodiments, two or more bone ties 100, 200, 300 and the interbody device 400 can limit or reduce motion of adjacent vertebrae. In some embodiments, the bone tie 100, 200, 300 and the interbody device 400 can limit motion to a range depending on the tightening of the loop of the bone tie 100, 200, 300. In some methods of use, the bone tie 100, 200, 300 and the interbody device 400 promote fusion of adjacent vertebrae.

The bone tie 100, 200, 300 can be configured to form a loop around at least a portion of the anatomy. The bone tie 100, 200, 300 can be disposed around a portion of the inferior vertebra. The bone tie 100, 200, 300 can be disposed around a portion of the superior vertebra. In some methods, a first bone tie 100, 200, 300 can be disposed around a portion of the inferior vertebra and a second bone tie 100, 200, 300 can be disposed around a portion of the superior vertebra. The bone tie 100, 200, 300 can be disposed through a portion of the vertebra. The bone tie 100, 200, 300 can be disposed through the lumen of the superior vertebra. The bone tie 100, 200, 300 can be disposed through the lumen of the inferior vertebra.

The bone tie 100, 200, 300 can be utilized alone. The bone tie 100, 200, 300 can be utilized in connection with another bone tie 100, 200, 300. The bone tie 100, 200, 300 can be utilized in connection with an interbody device 400. The bone tie 100, 200, 300 and the interbody device 400 can be utilized in connection with fusion material. The bone tie 100, 200, 300 and the interbody device 400 can be utilized in connection with bone grafts. The bone tie 100, 200, 300 and the interbody device 400 can be utilized in connection with any substance. The bone tie 100, 200, 300 and the interbody device 400 can be utilized in connection with any biologic and/or chemical substance, including, but not limited to, medicine, adhesives, etc., and/or a bone graft, including, but not limited to, autograft, allograft, xenograft, alloplastic graft, a synthetic graft, and/or combinations of grafts, medicines, and/or adhesives. While exemplary references are made with respect to vertebra, in some embodiments another bone can be involved. While specific reference may be made to a specific vertebra and/or subset and/or grouping of vertebrae, it is understood that any vertebra and/or subset and/or grouping, or combination of vertebrae can be used. The bone tie 100, 200, 300 and the interbody device 400 can deliver a substance. The interbody device 400 can be packed with a substance. The lumen of the vertebra that the bone tie 100, 200, 300 extends through can be packed with a substance. The bone tie 100, 200, 300 and the interbody device 400 can be configured to retain, carry and/or otherwise deliver a substance to aid in fusion, such as, for example, medicines, adhesives, bone graft, and/or combinations of substances.

The bone tie 100, 200, 300 can have several advantages. The bone tie 100, 200, 300 can allow for simplified subsequent removal techniques versus traditional hardware. The bone tie 100, 200, 300 can be easily cut to be removed. The bone tie 100, 200, 300 can be removed after fusion. The bone tie 100, 200, 300 can be adjusted during a procedure to adjust the tension on the underlying anatomy. The bone tie 100, 200, 300 can be adjusted during a procedure to increase or decrease the tension on the underlying anatomy. The bone tie 100, 200, 300 can be removed during a procedure to adjust the tension on the underlying anatomy. The bone tie 100, 200, 300 can be removed during a procedure to decrease the tension on the underlying anatomy. The bone tie 100, 200, 300 can removed and replaced with another bone tie 100, 200, 300. The bone tie 100, 200, 300 can absorb over time within the body of the patient. The bone tie 100, 200, 300 can be advantageously tightened in one direction. The bone tie 100, 200, 300 can maintain the tension under normal anatomical loads.

The bone tie 100, 200, 300 and the interbody device 400 can be utilized to correct or improve the condition of a variety of ailments. The bone tie 100, 200, 300 and the interbody device 400 can be utilized to correct or improve the condition of a coronal plane deformity. The bone tie 100, 200, 300 and the interbody device 400 can be utilized to correct or improve the condition of a lateral scoliosis. The bone tie 100, 200, 300 and the interbody device 400 can be utilized to achieve rotational correction. The bone tie 100, 200, 300 and the interbody device 400 can be utilized to achieve sagittal correction. The bone tie 100, 200, 300 and the interbody device 400 can be utilized to restore lordosis. The bone tie 100, 200, 300 can be tensioned to set sagittal correction. The bone tie 100, 200, 300 can be tensioned to set lordosis.

The bone tie 100, 200, 300 and the interbody device 400 can be utilized in combination with navigation systems to achieve desired trajectories. The bone tie 100, 200, 300 and the interbody device 400 can be utilized in combination with guidance systems to achieve desired trajectories. The bone tie 100, 200, 300 and the interbody device 400 can be utilized in combination with probes to achieve desired trajectories. The probe can facilitate forming an operative channel through the tissue of a patient to access a portion of the patient's spine. In operation, the probe can be inserted into a patient. In some embodiments, the probe is inserted into an anchorable location, such as a collagenous tissue, bone, or vertebral disc. In some embodiments, the probe comprises at least one electrode. In some embodiments, the at least one electrode is capable of stimulating a nerve to provoke an electromyographic response in the nerve.

In some embodiments described herein, the bone tie 100, 200, 300 and the interbody device 400 can be used to stabilize and/or fixate a first vertebra to a second vertebra. The bone tie 100, 200, 300 and the interbody device 400 can be configured to reduce pain associated with a bone portion. The bone tie 100 and the interbody device 400 can be configured to reduce further degradation of a spine. The bone tie 100, 200, 300 and the interbody device 400 can be configured to reduce further degradation of a specific vertebra of a spine. The bone tie 100, 200, 300 and the interbody device 400 can be configured to reduce movement until the first vertebra and the second vertebra have fused. The bone tie 100, 200, 300 and the interbody device 400 can be configured to stabilize the first vertebra and second vertebra by securing the first vertebra to the second vertebra. In some embodiments described herein, the bone tie 100, 200, 300 provides temporary fixation. In some embodiments described herein, the bone tie 100, 200, 300 provides permanent fixation. In some embodiments described herein, the bone tie 100, 200, 300 provides stabilization of the spine. In some embodiments described herein, the bone tie 100, 200, 300 can stabilize the operative site during healing. In some embodiments described herein, the bone tie 100, 200, 300 can be removed after fusion.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. For all the embodiments described above, the steps of the methods need not be performed sequentially. Thus, it is intended that the scope of the present invention herein disclosed should not be limited by the particular disclosed embodiments described above, but should be determined only by a fair reading of the claims that follow.

1. A method of treating a patient, the method comprising:
positioning an interbody device in an intervertebral space;
positioning a bone tie through the interbody device, the bone tie comprising a distal end and a fastener section; and
tightening the bone tie by passing the distal end of the bone tie through the fastener section of the bone tie.

2. The method of claim 1, further comprising forming a lumen in a vertebra.

3. The method of claim 2, further comprising positioning the bone tie through the lumen.

4. The method of claim 1, further comprising forming a lumen in a superior vertebra and an inferior vertebra.

5. The method of claim 4, further comprising positioning the bone tie through the lumen in the superior vertebra.

6. The method of claim 5, further comprising positioning a second bone tie through the interbody device and through the lumen in the inferior vertebra.

7. The method of claim 1, wherein positioning the bone tie through the interbody device further comprises passing the bone tie through a graft chamber.

8. The method of claim 1, wherein positioning the bone tie through the interbody device comprises passing the bone tie through a window.

9. The method of claim 1, further comprising packing a window of the interbody device with graft material.

10. The method of claim 9, wherein the bone tie is in communication with graft material.

11. The method of claim 9, wherein the bone tie is not in communication with graft material.

12. The method of claim 1, wherein positioning the bone tie through the interbody device comprises passing the bone tie through a notch.

13. The method of claim 1, wherein positioning the bone tie through the interbody device comprises passing the bone tie toward a lumen in a vertebra.

14. The method of claim 1, wherein tightening the bone tie comprises engaging gears of a bone tie with a ratchet.

15. The method of claim 1, wherein the interbody device is retained within a loop of the bone tie.

16. The method of claim 1, wherein tightening the bone tie comprises pulling the distal end through the fastener section to make a loop consecutively smaller.

17. The method of claim 1, further comprising packing the interbody device with graft material.

18. The method of claim 1, further comprising packing the interbody device after the bone tie is positioned through the interbody device.

19. The method of claim 1, further comprising packing the interbody device before the bone tie is positioned through the interbody device.

20-40. (canceled)
